(19) United States
(12) Patent Application Publication
(10) Pub. No.: US 2006/0110352 A1
(43) Pub. Date: May 25, 2006

(54) Cosmetic, pharmaceutical and dermatological compositions

(75) Inventors: Robert Milbradt, Wiesbaden (DE); Wibke Stelter, Grosskarben (DE); Michael Hornung, Frankfurt (DE); Sebastiano Lo Vasco, Friedberg (DE)

Correspondence Address: CLARIANT CORPORATION;INTELLECTUAL PROPERTY DEPARTMENT, 4000 MONROE ROAD, CHARLOTTE, NC, 28205, US

(73) Assignee: Clariant GmbH

(21) Appl. No.: 11/250,756

(22) Filed: Oct. 14, 2005

(30) Foreign Application Priority Data
Oct. 15, 2004 (DE) 102004050239.0

(51) Int. Cl. A61K 8/81

(52) U.S. Cl. 424/70.15; 424/70.16

(57) ABSTRACT

A description is given of cosmetic, dermatological and/or pharmaceutical compositions comprising at least one copolymer A containing a) 1% to 50% by weight of structural units originating from N-vinylcaprolactam,
b) 49.99% to 98.99% by weight of repeating structural units of the formula (1) 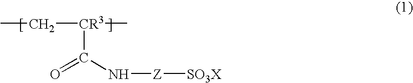 in which
R³ is hydrogen, methyl or ethyl,
Z is C₁-C₈ alkylene and
X⁺ is Li⁺, Na⁺, K⁺, Mg²⁺/2, Ca²⁺/2, Al³⁺/3, NH₄₊, monoalkylammonium, dialkylammonium, trialkylammonium or tetraalkylammonium, the alkyl substituents of the ammonium ions being independently of one another (C₁-C₂₂) alkyl radicals or (C₂-C₁₀) hydroxyalkyl radicals, or singly to triply ethoxylated ammonium compounds having the same or different degree of ethoxylation, it being possible also for two or more different structural units of the formula (1) to be present in the copolymer, and c) 0.01% to 8% by weight, preferably 0.01% to 5% by weight, of crosslinking structures originating from monomers having at least two olefinic double bonds.

The present invention relates in particular to cosmetic, pharmaceutical, and dermatological compositions comprising comb copolymers of the monomers of acryloyidimethyltaurine and/or salts thereof and N-vinylcaprolactam.

Consumer requirements and cosmetic product rheology are closely interlinked. Thus, for example, the visual appearance of a cream or lotion is influenced by its viscosity. The sensorial properties, such as consistency or spreadability, determine the individual profile of a cosmetic product. The effectiveness of active substances (e.g., sunscreen filters) and the storage stability of the formulation are also closely related to the rheological properties of the products.

In the cosmetics sector a leading part is played by polyelectrolytes as thickeners and gel formers. State of the art are, in particular, thickeners based on poly(meth)acrylic acid and the water-soluble copolymers thereof. The diversity of the possible structures and the diverse possibilities for use that are associated therewith are manifested not least in a multiplicity of patents filed worldwide since the mid-1970s.

A substantial drawback of thickeners based on poly(meth)acrylic acid is the heavy pH dependency of the thickening performance. Thus, in general, a sufficiently high viscosity is developed only when the pH of the formulation is adjusted to a level of more than 6, i.e., the poly(meth)acrylic acid is in neutralized form. Furthermore, the corresponding compositions are sensitive to UV radiation and also to shearing, and they also impart a sticky feeling on the skin. The handling of such thickeners is also problematic. Since the thickeners are generally in an acidic form, formulation involves an additional neutralizing step.

In the 1990s, innovative thickeners based on crosslinked and neutralized polyacryloyldimethyltaurates were introduced into the market (EP 0 815 828, EP 0 815 844, EP 0 815 845 and EP 0 850 642). EP 1 028 129 and EP 1 116 733 describe copolymers of acryloyldimethyltaurine and/or salts thereof and linear N-vinylcarboxamides and, respectively, copolymers of acryloyldimethyltaurine and/or salts thereof and linear N-vinylcarboxamides and cyclic vinylcarboxamides (Aristoflex® AVC, Clariant GmbH), as thickeners. These acryloyldimethyltaurate-based thickener systems display good properties in pH ranges below 6, and also a high UV stability and shearing stability, outstanding viscoelastic properties, great ease of processing, and a favorable toxicological profile.

There is, nevertheless, great interest in developing thickeners combining improved emulsification, dispersing, and gelling with a very low use concentration, said thickeners giving the compositions a clear, esthetic appearance, exhibiting high compatibility with additives, producing good skin sensation, and being stable on storage.

Surprisingly it has found that this object is achieved by copolymers A containing
a) 1 % to 50% by weight of structural units originating from N-vinylcaprolactam,
b) 49.99% to 98.99% by weight of repeating structural units of the formula (1) 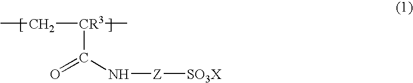
in which
R³ is hydrogen, methyl or ethyl,
Z is C₁-C₈ alkylene and
X⁺ is Li⁺, Na⁺, K⁺, Mg²⁺/2, Ca²⁺/2, Al³⁺/3, NH₄₊, monoalkylammonium, dialkylammonium, trialkylammonium or tetraalkylammonium, the alkyl substituents of the ammonium ions being independently of one another (C₁-C₂₂) alkyl radicals or (C₂-C₁₀) hydroxyalkyl radicals, or singly to triply ethoxylated ammonium compounds having the same or different degree of ethoxylation,
   it being possible also for two or more different structural units of the formula (1) to be present in the copolymer, and
c) 0.01% to 8% by weight, preferably 0.01% to 5% by weight of crosslinking structures originating from monomers having at least two olefinic double bonds.

The invention accordingly provides cosmetic, dermatological and/or pharmaceutical compositions comprising at least one copolymer A containing
a) 1% to 50% by weight of structural units originating from N-vinylcaprolactam,
b) 49.99% to 98.99% by weight of repeating structural units of the formula (1) 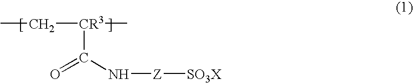
   in which
   R³ is hydrogen, methyl or ethyl,
   Z is C₁-C₈ alkylene and
   X⁺ is Li⁺, Na⁺, K⁺, Mg²⁺/2, Ca²⁺/2, Al³⁺/3, NH₄+, monoalkylammonium, dialkylammonium, trialkylammonium or tetraalkylammonium, the alkyl substituents of the ammonium ions being independently of one another (C₁-C₂₂) alkyl radicals or (C₂-C₁₀) hydroxyalkyl radicals, or singly to triply ethoxylated ammonium compounds having the same or different degree of ethoxylation,
   it being possible also for two or more different structural units of the formula (1) to be present in the copolymer, and
c) 0.01% to 8% by weight, preferably 0.01% to 5% by weight of crosslinking structures originating from monomers having at least two olefinic double bonds.

The copolymers A exhibit improved emulsifying and gelling capacity at use concentrations reduced in comparison to Aristoflex® AVC, and, even in acidic compositions, especially preparations containing hydroxy acid, have an excellent thickener performance and result in effective stabilization of emulsions on an oil-in-water, water-in-oil, and water-in-silicone basis.

The copolymers A have a very good and rapid emulsifying capacity, are highly suitable for adjusting viscosity, even of electrolyte-containing compositions, enhance the storage stability and temperature stability of the emulsions or dispersions, and produce a good feeling of freshness on the skin. The copolymers A exhibit excellent dispersing capacity for solid constituents in liquid or paste compositions and enhance the compatibility of individual components customary in cosmetic and pharmaceutical compositions.

They can therefore be used with advantage in, for example, decorative compositions, sun protection products, and deodorants, and also in hair coloring, styling, and care compositions.

With great advantage the copolymers A can be used to produce sprayable, pumpable, and foamable, non-aerosol gels and foams, especially sprayable hair gels and foamable sun protection products with optimized spray pattern and optimized droplet size distribution.

Preferred copolymers A contain 2% to 30%, preferably 3% to 15% by weight of structural units originating from N-vinylcaprolactam, 69.5% to 97.5%, preferably 84.5% to 96.5% by weight of structural units of the formula (1), derived in particular from the ammonium salt of 2-acrylamido-2-methylpropanesulfonic acid, and 0.2% to 4%, preferably 0.5% to 3% by weight of crosslinking structures originating from monomers having at least two olefinic double bonds.

Crosslinking structures originating from monomers having at least two olefinic double bonds derive preferably from allyl acrylates or methacrylates, dipropylene glycol diallyl ether, polyglycol diallyl ether, triethylene glycol divinyl ether, hydroquinone diallyl ether, tetraallyloxyethane or other allyl or vinyl ethers of polyfunctional alcohols, tetraethylene glycol diacrylate, triallylamine, trimethylolpropane diallyl ether, methylenebisacrylamide, trimethylolpropane triacrylate, trimethylolpropane trimethacrylate or divinylbenzene.

In one preferred embodiment the crosslinking structures derive from monomers of the formula (2) 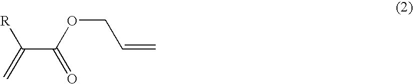
in which R is hydrogen, methyl or ethyl.

In another preferred embodiment the crosslinking structures are structures originating from trimethylolpropane triacrylate of the formula (3).
H₅C₂—C[CH₂OOC—CH═CH₂]₃ (3)

The copolymers A are prepared by dissolving or dispersing the corresponding monomers a) and b) in a protic solvent, adding to this solution or dispersion one or more crosslinkers having at least two olefinic double bonds, and initiating the polymerization in conventional fashion by adding a compound which forms free radicals.

Preference is given to copolymerizing the ammonium salt of acrylamidopropylsulfonic acid. Instead of this ammonium salt it is also possible to use the free acrylamidopropylsulfonic acid and, before adding the remaining monomers, to generate the ammonium salt from the free acid by introducing ammonia.

In another preferred embodiment the copolymers A are composed essentially of components a), b), and c).

In another preferred embodiment the compositions of the invention contain, based on the total weight of the compositions, 0.01% to 10% by weight, preferably 0.1% to 5% by weight, more preferably 0.5% to 3% by weight of copolymers A.

The polymerization reaction takes place preferably in a water-soluble alcohol or in a mixture of two or more alcohols having 1 to 6 carbon atoms, preferably in tert-butanol. The water content of the alcohol or of the mixture of two or more alcohols must not exceed 10% by weight, since otherwise-lumps may be formed in the course of the polymerization. Specifically, the choice of the identity and amount of the solvent must take place such that the salt of acrylamidoalkylsulfonic acid of the formula (1), in particular of 2-acrylamido-2-methylpropanesulfonic acid, is largely soluble or dispersible in said solvent. Largely soluble or dispersible means that, even after the stirrer has been shut off, no solid material settles out of the solution or dispersion. The polymer formed in the course of the reaction, in contrast, should be largely insoluble in the chosen solvent or solvent mixture. By largely insoluble is meant in this context that, in the course of the polymerization, a readily stirrable, porridgy polymer paste develops in which there must be no lumps or sticky concretions formed.

The filtrate obtainable by filtering the paste with suction must not have a solids content of more than 5% by weight. If the copolymers are soluble to a greater extent in the chosen solvent or solvent mixture, lumping may occur when the polymer paste is dried.

The polymerization reaction itself is initiated in conventional fashion by means of free radical-forming compounds such as azo initiators (e.g., azobisisobutyronitrile), peroxides (e.g., dilauroyl peroxide) or persulfates in an appropriate temperature interval from 20 to 120° C., preferably between 40 and 80° C., and is continued over a period of 30 minutes to several hours.

The copolymer composition can be varied by varying the above-described ratio of the monomers used and also the fraction of crosslinker and using them thus to produce a custom-tailored profile of properties. Through increased incorporation of ammonium salts of acrylamidosulfonic acids, for example, the thickening action of the polymers can be improved, while by incorporating more N-vinylcaprolactam it is possible to improve the electrolyte compatibility of the polymers and their solubility in nonaqueous systems.

In another preferred embodiment the compositions of the invention have a pH of less than 6 and as well as the copolymers A contain organic or inorganic acids, preferably organic acids, more preferably α- or β-hydroxy acids.

Particular preference is given to a-hydroxy acids of the formula (4) 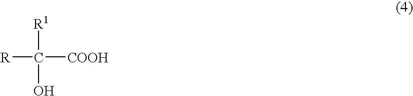
where R and R¹ independently of one another can be H, F, Cl, Br, alkyl, aralkyl or aryl groups with saturated or unsaturated, linear or branched chains, cyclic groups or OH, CHO, COOH or alkoxy groups having 1 to 9 carbon atoms. Use may be made of the acid and/or of the corresponding salt with alkali metal or ammonium counterions.

Suitable acidic components include glycolic acid, lactic acid, methyllactone acid, 2-hydroxybutanoic, -pentanoic, -hexanoic, -heptanoic, -octanoic, -nonanoic, -decanoic, -undecanoic, and -dodecanoic acid (laurylic acid), α-hydroxymyristic acid, α-hydroxypalmitic acid, α-hydroxystearic acid, arachidonic acid, 2-phenyl-2-hydroxyethanoic acid, citric acid, tartaric acid, mandelic acid, salicylic acid, ascorbic acid, pyruvic acid, 2,2-diphenyl-2-hydroxyethanoic acid, 3-phenyl-2-hydroxypropanoic acid, 2-phenyl-2-methyl-2-hydroxyethanoic acid, 2-(4′-hydroxyphenyl)-2-hydroxyethanoic acid, 2-(4′-dichlorophenyl)-2-hydroxyethanoic acid, 2-(3′-hydroxy-4′-methoxyphenyl)-2-hydroxyethanoic acid, 2-(4′-hydroxy-3′-methoxyphenyl)-2-hydroxyethanoic acid, 3-(2′-hydroxyphenyl)-2-hydroxypropanoic acid, 3-(4′-hydroxyphenyl)-2-hydroxypropanoic acid, 2-(3′,4′-dihydroxyphenyl)-2-hydroxyethanoic acid, fumaric acid, retinoic acid, aliphatic and organic sulfonic acids, benzoic acid, kojic acid, fruit acid, malic acid, gluconic acid, galacturonic acid, acidic plant extracts and/or fruit extracts, and derivatives thereof.

In a further preferred embodiment the compositions contain, based on the total weight of the compositions, 0.01% to 20% by weight, preferably 0.5% to 10% by weight and more preferably 1% to 5% by weight of hydroxy acids, preferably α-hydroxy acids, which may also be present partly in salt form. In one particularly preferred embodiment these compositions contain, based on the total weight of the compositions, 0.01% to 10% by weight of copolymer A.

With particular preference the compositions of the invention comprise glycolic acid, lactic acid and/or 2-hydroxyoctanoic acid.

The hydroxy acid and the corresponding salt are preferably in a molar ratio in the range from 1000:1 to 1:1000, more preferably 50:1 to 1:50.

Further preferred embodiments of the compositions of the invention are liquid/liquid dispersions comprising an aqueous phase and an oily phase, especially water-in-oil emulsions, oil-in-water emulsions, oil-in-water microemulsions, and multiple emulsions, gel creams, and solid/liquid dispersions.

The emulsions can be prepared in known fashion, i.e., for example, by hot, hot/cold or PIT emulsification.

The copolymers A have a very good and rapid emulsifying capacity, are highly suitable for viscosity adjustment, enhance the storage stability and temperature stability of the emulsions or dispersions, and produce a good feeling of freshness on the skin.

The copolymers A display an excellent dispersing capacity for solid constituents in liquid or paste compositions and enhance the compatibility of individual components. They can therefore be used with advantage in, for example, decorative compositions, sun protection products, and deodorants.

The copolymers A can be used with very great advantage to produce pumpable and foamable, non-aerosol gels and foams, especially sprayable hair gels and foamable sun protection products.

In further preferred embodiments the compositions of the invention comprise water, oil, one or more emulsifiers, and one or more copolymers A.

Where the compositions of the invention constitute emulsions, the oil phase may be selected advantageously from the groups of mineral oils, mineral waxes, oils, such as triglycerides, fats, waxes, and other natural and synthetic fatty substances, preferably esters of fatty acids with alcohols of low carbon number, e.g., with isopropanol, propylene glycol or glycerol, or esters of fatty alcohols with alkanoic acids of low carbon number with fatty acids, alkyl benzoates.

One class of inventively preferred oils and fats are the triglycerides of linear or branched, saturated or unsaturated, optionally hydroxylated C₈-C₃₀ fatty acids, especially vegetable oils, such as sunflower oil, corn oil, soybean oil, rice oil, jojoba oil, babusscu oil, pumpkin oil, grapeseed oil, sesame oil, walnut oil, apricot oil, orange oil, wheatgerm oil, peach kernel oil, macadamia oil, avocado oil, sweet almond oil, lady's-smock oil, castor oil, olive oil, peanut oil, rapeseed oil, and coconut oil, and also synthetic triglyceride oils, e.g., the commercial product Myritol®318. Hydrogenated triglycerides as well are inventively preferred. It is also possible to use oils of animal origin, examples being bovine tallow, perhydrosqualene, and lanolin.

A further class of inventively preferred oils and fats are the benzoic esters of linear or branched C₈₋₂₂ alkanols, e.g., the commercial products Finsolv®SB (isostearyl benzoate), Finsolv®TN (C₁₂-C₁₅ alkyl benzoate) and Finsolv®EB (ethylhexyl benzoate).

A further class of inventively preferred oils and fats are the dialkyl ethers with in total 12 to 36 carbon atoms, especially 12 to 24 carbon atoms, such as e.g. di-n-octyl ether (Cetiol® OE), di-n-decyl ether, di-n-nonyl ether, di-n-undecyl ether, di-n-dodecyl ether, n-hexyl n-octyl ether, n-octyl n-decyl ether, n-decyl n-undecyl ether, n-undecyl n-dodecyl ether, and n-hexyl n-undecyl ether, and also di-tert-butyl ether, diisopentyl ether, di-3-ethyldecyl ether, tert-butyl n-octyl ether, isopentyl n-octyl ether and 2-methylpentyl n-octyl ether.

An additional class of inventively preferred oils and fats are hydrocarbon oils: for example, those with linear or branched, saturated or unsaturated C₇-C₄₀ carbon chains, examples being vaseline, dodecane, isododecane, cholesterol, lanolin, hydrogenated polyisobutylenes, docosanes, hexadecane, isohexadecane, liquid paraffins, paraffin waxes, isoparaffin oils, e.g., the commercial products of the Permethyl® series, squalane, squalene, synthetic hydrocarbons such as polyisobutene, and alicyclic hydrocarbons, e.g., the commercial product 1,3-di-(2-ethylhexyl)cyclohexane (Cetiol®S), ozokerite, microwaxes, and ceresin.

Likewise suitable are branched saturated or unsaturated fatty alcohols having 6-30 carbon atoms, e.g., isostearyl alcohol, and also guerbet alcohols.

Another class of inventively preferred oils and fats are hydroxycarboxylic acid alkyl esters. Preferred hydroxycarboxylic acid alkyl esters are full esters of glycolic acid, lactic acid, malic acid, tartaric acid or citric acid. Further hydroxycarboxylic esters suitable in principle are esters of β-hydroxypropionic acid, of tartronic acid, of D-gluconic, saccharic acid, mucic acid or glucuronic acid. Suitable alcohol components of these esters include primary, linear or branched aliphatic alcohols having 8 to 22 carbon atoms. The esters of C₁₂-C₁₅ fatty alcohols are particularly preferred. Esters of this type are available commercially: for example, under the trade name Cosmacol® from EniChem, Augusta Industriale.

A further class of inventively preferred oils and fats are dicarboxylic esters of linear or branched C₂-C₁₀ alkanols, such as di-n-butyl adipate (Cetiol®B), di(2-ethylhexyl) adipate, di(2-ethylhexyl) succinate and diisotridecyl azelaate, and also diol esters such as ethylene glycol dioleate, ethylene glycol diisotridecanoate, propylene glycol di(2-ethylhexanoate), propylene glycol diisostearate, propylene glycol dipelargonate, butanediol diisostearate, and neopentyl glycol dicaprylate.

Likewise preferred oils and fats are symmetrical, asymmetrical or cyclic esters of carbonic acid with fatty alcohols, glyceryl carbonate or dicaprylyl carbonate (Cetiol®CC).

A further class of inventively preferred oils and fats are the esters of dimers of unsaturated C₁₂-C₂₂ fatty acids (dimer fatty acids) with monohydric linear, branched or cyclic C₂-C₁₈ alkanols or with polyhydric linear or branched C₂-C₆ alkanols.

In a further preferred embodiment of the invention the compositions of the invention are in the form of a water-in-silicone emulsion and comprise water, silicone, one or more emulsifiers, and one or more copolymers A.

Silicone oils and silicone waxes preferably available are dimethylpolysiloxanes and cyclomethicones, polydialkylsiloxanes R₃SiO(R₂SiO)ₓSiR₃, where R is methyl or ethyl, more preferably methyl, and x is a number from 2 to 500, examples being the dimethicones available under the trade names VICASIL (General Electric Company), DOW CORNING 200, DOW CORNING 225, DOW CORNING 200 (Dow Corning Corporation), and also the dimethicones available under SilCare® Silicone 41 M65, SilCare® Silicone 41 M70, SilCare® Silicone 41M80 (Clariant GmbH), stearyldimethylpolysiloxane, C₂₀-C₂₄ alkyl-dimethylpolysiloxane, C₂₄-C₂₈ alkyl-dimethylpolysiloxane, and also the methicones available under SilCare® Silicone 41M40, SilCare® Silicone 41M50 (Clariant GmbH), and additionally trimethylsiloxysilicates [(CH₂)₃SiO)_{1/2}]ₓ[SiO₂]_{y}, where x is a number from 1 to 500 and y is a number from 1 to 500, dimethiconols R₃SiO[R₂SiO]ₓSiR₂OH and HOR₂SiO[R₂SiO]ₓSiR₂OH, where R is methyl or ethyl and x is a number up to 500, polyalkylarylsiloxanes, examples being the polymethylphenylsiloxanes available under the commercial designations SF 1075 METHYLPHENYL FLUID (General Electric Company) and 556 COSMETIC GRADE PHENYL TRIMETHICONE FLUID (Dow Corning Corporation), polydiarylsiloxanes, silicone resins, cyclic silicones and amino-, fatty acid-, alcohol-, polyether-, epoxy-, fluoro- and/or alkyl-modified silicone compounds, and also polyethersiloxane copolymers.

Suitable emulsifiers include nonionic, anionic, and ampholytic surface-active agents.

Nonionic emulsifiers available are adducts of 0 to 30 mol of ethylene oxide and/or 0 to 5 mol of propylene oxide with linear fatty alcohols having 6 to 30 carbon atoms, preferably 10 to 22 carbon atoms, and very preferably 14 to 22 carbon atoms, optionally in hydroxylated form. Use may be made, for example, of octanol (caprylyl alcohol), octenol, octadienol, decanol (capryl alcohol), decenol, decadienol, dodecanol (lauryl alcohol), dodecenol, dodecadienol, ricinoleyl alcohol, myristyl alcohol, cetyl alcohol, stearyl alcohol, cetearyl alcohol, oleyl alcohol, linoleyl alcohol, linolenyl alcohol, arachidyl alcohol, and behenyl alcohol. Use may also be made of fatty alcohol cuts produced by reducing naturally occurring triglycerides, such as bovine tallow, palm oil, peanut oil, colza oil, cottonseed oil, soybean oil, sunflower oil, and linseed oil, or fatty acid esters produced from transesterification products with corresponding alcohols, and which therefore constitute a mixture of different fatty alcohols. Substances of this kind are available commercially, for example, under the designation Stenol®, e.g., Stenol® 1618 or Lanette®, e.g., Lanette® O and Lanette®22, or Lorol®, e.g., Lorol®C18.

Also suitable are lanolin fats.

Particular preference is given to cetyl alcohol, stearyl alcohol, cetearyl alcohol, arachidyl alcohol, and behenyl alcohol.

A further class of inventively preferred emulsifiers are adducts having 0 to 30 mol of ethylene oxide and/or 0 to 5 mol of propylene oxide with linear and/or branched, saturated and/or unsaturated fatty acids having 6 to 30 carbon atoms, preferably 10 to 22 carbon atoms. Mention may be made of isostearic acid, such as the commercial products Emersol®871 and Emersol®875, isopalmitic acids such as Edenor®IP95, and all other fatty acids which can be purchased under the commercial designation Edenor® (Cognis). Further typical examples of such fatty acids are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid, erucic acid, and dimers of unsaturated fatty acids, and also their technical mixtures. Particular preference is given to fatty acid cuts from coconut oil or palm oil; stearic acid is especially preferred.

Normally the fatty acids are neutralized with a basic agent, NaOH for example, and are used in the form, for example, of their sodium, potassium, ammonium, calcium, magnesium, and zinc salts.

A further class of emulsifiers which can be used are esters of sugars—alkylated if desired—with C₆-C₃₀ fatty acids. Sugars which can be used are any monosaccharides or oligosaccharides. It is usual to use monosaccharides with 5 or 6 carbon atoms, examples being ribose, xylose, lyxose, altose, glucose, fructose, galactose, arabinose, altrose, mannose, gulose, idose, and talose, and also the deoxy sugars rhamnose and fucose. Sugars with 4 carbon atoms as well can be used, e.g., erythrose and threose. Inventively suitable oligosaccharides are composed of two to 10 monosaccharide units, e.g., sucrose (saccharose), lactose or trehalose. Preferred sugar units are the monosaccharides glucose, fructose, galactose, arabinose, and the disaccharide sucrose; glucose and sucrose are particularly preferred. The sugars may have been partially etherified with methyl, ethyl, propyl, isopropyl or butyl groups, e.g., methylglucoside, ethylglucoside or butylglucoside. For esterification it is possible to use all C₆-C₃₀ fatty acids and mixtures thereof which were cited above. Also suitable in principle are mono- and polyesterified sugars; preference is given to the monoesters, sesquiesters and diesters, examples being sucrose monostearate, sucrose distearate, sucrose monococoate, sucrose dicocoate, methylglucoside monostearate, methylglucoside sesquistearate, methylglucoside isostearate, ethylglucoside monolaurate, ethylglucoside dilaurate, ethylglucoside monococoate, ethylglucoside dicocoate and butylglucoside monococoate.

Another class of suitable emulsifiers are C₈-C₂₂ alkyl-monoglycosides and -oligoglycosides, alkyl-mono- and -oligoglycosides conforming to the formula RO-(Z)ₓ, where R is a C₈-C₂₂ alkyl group, Z is the sugar, and x is the number of sugar units. The alkyl-mono- and -oligoglycosides which can be used in accordance with the invention may contain only one particular alkyl radical R. Particular preference is given to those alkyl-mono- and -oligoglycosides in which R is composed essentially of C₈ and C₁₀ alkyl groups, essentially of C₁₂ and C₁₄ alkyl groups, essentially of C₈ to C₁₆ alkyl groups, or essentially of C₁₂ to C₁₆ alkyl groups. As sugar unit Z it is possible to use any desired monosaccharides or oligosaccharides, such as those mentioned above. Preferred sugar units are glucose, fructose, galactose, arabinose, and sucrose, with glucose being particularly preferred. The alkyl-mono- and -oligoglycosides which can be used in accordance with the invention contain on average 1.1-5, preferably 1.1-2.0, and more preferably 1.1-1.8 sugar units. The alkoxylated homologs of the stated alkyl-mono- and -oligoglycosides can also be used in accordance with the invention. These homologs may contain on average up to 10 ethylene oxide and/or propylene oxide units per alkylglycoside unit. Suitability is possessed for example by cocoylglucoside, decylglucoside, laurylglucoside, cetarylglucoside, and arachidylglucoside. Also of particular preference in addition to the stated alkyl-mono- and -oligoglucosides are the mixtures of alkyl-mono- and -oligoglucosides and fatty alcohols, examples being the commercially available products Montanov®68 and Montanov®202.

A further class of suitable emulsifiers are the partial esters of propylene glycol, glycerol, and sorbitan with C₈-C₂₂ fatty acids. For esterification it is possible to use all C₈-C₂₂ fatty acids and mixtures thereof, as have already been cited above. Particularly suitable examples are propylene glycol monostearate, glyceryl monolaurate, glyceryl monostearate, glyceryl distearate, glyceryl monooleate, sorbitan monolaurate, sorbitan dilaurate, sorbitan monostearate, sorbitan sesquistearate, sorbitan distearate, sorbitan monoisostearate, sorbitan monooleate, and sorbitan dioleate, or the commercial products Monomuls®90-0, Monomuls®90-L 12 and Cutina®MD. These emulsifiers may contain on average up to 10 ethylene oxide and/or propylene oxide units per molecule.

A further preferred class of emulsifiers are polyglycerols of the formula HO—CH₂—CHOH—CH₂[—O—CH₂—CHOH—CH₂]ₙ—O—CH₂—CHOH—CH₂OH with n=0-8 and their esters with linear and branched C₈-C₂₂ fatty acids, which may carry functional groups in the alkyl chain, preferably polyglyceryl-2 dipolyhydroxystearate (commercial product Dehymuls® PGPH) and polyglyceryl-3 diisostearate (commercial product Lameform® TGI).

A further class of preferred emulsifiers are sterols, especially cholesterol, lanosterol, β-sitosterol, stigmasterol, campesterol, and ergosterol, and also mycosterols. Commercially customary sterol emulsifiers are prepared on the basis of soybean sterols or rapeseed sterols. Preference is given in accordance with the invention to the use of sterols containing 5-10 ethylene oxide units per molecule. Suitability is possessed for example by the commercial products Generol®122, Generol® 122 E 5, Generol® 122 E 10, and Generol®RE 10.

Emulsifiers which can also be used with preference are phospholipids, especially the phosphatidylcholines or lecithins. Phospholipids are phosphoric diesters, less often monoesters, of usually linear saturated and unsaturated C₈-C₂₂ fatty acids; soya lecithin is preferred.

A further class of preferred emulsifiers are the esterification products of lactic acid or glycolic acid with linear or branched C₈-C₂₂ fatty acids and also the sodium, potassium, ammonium, calcium, magnesium, and zinc salts of these esterification products.

Particularly preferred esterification products are those of the formula (5) 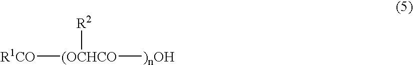
where R¹ is a linear or branched saturated or unsaturated alkyl radical having 5 to 21 carbon atoms and R² is a methyl group or a hydrogen atom, and n is an integer 1-4.

Among the acyl radicals R¹CO—, preference is given in turn to the radicals selected from the caprooyl, capryloyl, caproyl, lauroyl, myristoyl, cetoyl, palmitoyl, stearoyl, isostearoyl, and the oleyl group. The stearoyl and the isostearoyl groups are particularly preferred.

The radical R² is preferably methyl.

The degree of oligomerization, n, is preferably 1 or 2.

A particularly preferred compound is sodium stearoyl-2 lactylate.

A further class of emulsifiers used with preference are phosphoric monoesters, diesters, and triesters of saturated or unsaturated linear or branched fatty alcohols having 8 to 30 carbon atoms and their ethylene oxide adducts with 1-10 ethylene oxide groups per molecule. These alkyl and alkenyl phosphates are depicted in the formula (6) 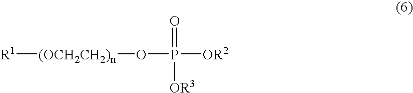
in which R¹ is a saturated or unsaturated, linear or branched hydrocarbon radical having 8 to 30 carbon atoms, R² and R³ independently of one another are a hydrogen atom, X or a radical (CH₂CH₂O)ₙR¹, n is numbers from 0 to 10, and X is an alkali metal or alkaline earth metal cation or a cation NR⁴R⁵R R⁷, where R⁴ to R⁷ independently of one another are a C₁-C₄ hydrocarbon radical.

The inventively preferred alkyl and alkenyl phosphates have as their group R¹ alkyl radicals having 12-18 carbon atoms, these radicals being saturated or unsaturated and linear or branched. These groups R¹ are, in particular, lauryl, myristyl, cetyl, palmityl, stearyl, isostearyl, and oleyl. Preferred values for n are either 0 or values 1 -10, preferably 2-5, more preferably 3-4 (alkyl or alkenyl ether phosphates). Preference is further given to the use of ester mixtures of monoesters, diesters, and triesters, the fraction of monoester and diester being predominant over the triester fraction. The use of pure triesters, however, may likewise be preferred. Suitable commercial products come from the Hostaphat® series (Clariant), e.g., Hostaphat®KW 340 D, Hostaphat®KO300 N, Hostaphat®KO380, and Hostaphat®KL 340.

Another class of emulsifiers used with preference in accordance with the invention are acylglutamates of the formula (7) 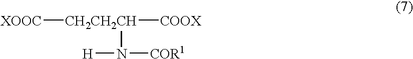
in which R¹CO is a linear or branched acyl radical having 6 to 22 carbon atoms and 0, 1, 2 or 3 double bonds and X is hydrogen, an alkali metal or alkaline earth metal cation, an ammonium, alkylammonium, alkanolammonium and/or glucammonium, examples being acylglutamates deriving from fatty acids having 6 to 22, preferably 12 to 18, carbon atoms, such as, for example, C_{12/14} and C_{12/18} coconut fatty acid, lauric acid, myristic acid, palmitic acid and/or stearic acid, particularly sodium N-cocoyl- and sodium N-stearoyl-L-glutamate.

A further class of inventively preferred emulsifiers are the esters

of a hydroxy-substituted dicarboxylic or tricarboxylic acid of the formula (8) 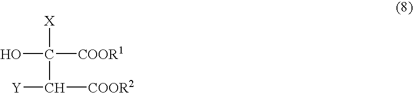
in which X is H or a —CH₂COOR group, Y is H or —OH, with the proviso that Y is H if X is —CH₂COOR, R, R¹ and R² independently of one another are a hydrogen atom, an alkali metal or alkaline earth metal cation, an ammonium group, the cation of an ammonium-organic base, or a radical Z that comes from a polyhydroxylated organic compound selected from the group of etherified (C₆-C₁₈)-alkylsaccharides with 1 to 6 monomeric saccharide units and/or of etherified aliphatic (C₆-C₁₆)-hydroxyalkylpolyols having 2 to 16 hydroxyl radicals, with the proviso that at least one of the groups, R, R¹ or R², is a radical Z.

A further class of inventively preferred emulsifiers are the esters of the sulfosuccinic salt of the formula (9) 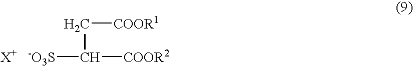
in which R¹ and R² independently of one another are a hydrogen atom, an alkali metal or alkaline earth metal cation, an ammonium group, the cation of an ammonium-organic base, or a radical Z that comes from a polyhydroxylated organic compound selected from the group of etherified (C₆-C₁₈)-alkylpolysaccharides having 1 to 6 monomeric saccharide units and/or of etherified aliphatic (C₆-C₁₈)-hydroxyalkylpolyols having 2 to 16 hydroxyl radicals, with the proviso that at least one of the groups, R¹ or R², is a radical Z, and X⁺ is an alkali metal or alkaline earth metal cation, an ammonium group or the cation of an ammonium-organic base.

A further class of inventively preferred emulsifiers are the sulfosuccinic monoalkyl and dialkyl esters having 8 to 24 carbon atoms in the alkyl group and sulfosuccinic monoalkylpolyoxy esters having 8 to 24 carbon atoms in the alkyl group and 1 to 6 ethoxy groups, and their alkali metal, alkaline earth metal or ammonium salts.

A further class of inventively preferred emulsifiers are the esters of tartaric acid and citric acid with alcohols which constitute adducts of about 2 to 10 molecules of ethylene oxide and/or propylene oxide with fatty alcohols having 8 to 22 carbon atoms, and their alkali metal, alkaline earth metal or ammonium salts.

Further inventively preferred emulsifiers are ethercarboxylic acids of the formula R—O—(CH₂—CH₂O)ₓ—CH₂—COOH, in which R is a linear alkyl group having 8 to 30 carbon atoms and x is 0 or 1 to 10, acylsarcosinates having a linear or branched acyl radical having 6 to 22 carbon atoms and 0,1, 2 or 3 double bonds, acyltaurates having a linear or branched acyl radical having 6 to 22 carbon atoms and 0, 1, 2 or 3 double bonds, and acylisethionates having a linear or branched acyl radical having 6 to 22 carbon atoms and 0, 1, 2 or 3 double bonds, and also the alkali metal, alkaline earth metal or ammonium salts of these emulsifiers.

The compositions of the invention may comprise mixtures of compounds from two or more of these classes of substance.

In one preferred embodiment the compositions of the invention are in the form of emulsions of the oil-in-water type, preferably in the form of cosmetic or dermatological emulsions of the oil-in-water type, and contain, based on the total weight of the compositions,
a) up to 95% by weight, preferably 60% to 92% by weight, more preferably 70% to 90% by weight, very preferably 75% to 85% by weight of a water phase,
b) up to 40% by weight, preferably 1% to 40% by weight, more preferably 2% to 25% by weight, very preferably 5% to 20% by weight of an oil phase,
c) up to 15% by weight, preferably 0.5% to 12% by weight, more preferably 1% to 8% by weight, very preferably 1% to 5% by weight of one or more emulsifiers, and
d) up to 5% by weight, preferably 0.01% to 5% by weight, more preferably 0.05% to 3% by weight, very preferably 0.1% to 2% by weight of copolymer A.

In a further preferred embodiment, the compositions of the invention are in the form of gel creams of the oil-in-water type, preferably in the form of cosmetic or dermatological gel creams of the oil-in-water type, and contain, based on the total weight of the compositions,
a) up to 95% by weight, preferably 50% to 95% by weight, more preferably 70% to 90% by weight, very preferably 75% to 85% by weight of a water phase,
b) up to 30% by weight, preferably 1% to 30% by weight, more preferably 3% to 25% by weight, very preferably 5% to 15% by weight of an oil phase,
c) up to 5% by weight, preferably 0.5% to 5% by weight, more preferably 0.2% to 4% by weight, very preferably 0.5% to 3% by weight of one or more emulsifiers, and
d) up to 5% by weight, preferably 0.01% to 5% by weight, more preferably 0.05% to 3% by weight, very preferably 0.1% to 2% by weight of copolymer A.

In a further preferred embodiment the compositions of the invention are in the form of emulsions of the water-in-oil type, preferably in the form of cosmetic or dermatological emulsions of the water-in-oil type, and contain, based on the total weight of the compositions,
a) up to 95% by weight, preferably 40% to 95% by weight, more preferably 50% to 90% by weight, very preferably 60% to 85% by weight of a water phase,
b) up to 60% by weight, preferably 2% to 60% by weight, more preferably 5% to 40% by weight, very preferably 10% to 30% by weight of an oil phase,
c) up to 20% by weight, preferably 0.5% to 20% by weight, more preferably 1% to 15% by weight, very preferably 4% to 12% by weight of one or more emulsifiers, and
d) up to 5% by weight, preferably 0.01% to 5% by weight, more preferably 0.05% to 3% by weight, very preferably 0.1% to 2% by weight of copolymer A.

In a further preferred embodiment the compositions of the invention are in the form of emulsions of the water-in-silicone type, preferably in the form of cosmetic or dermatological emulsions of the water-in-silicone type, and contain, based on the total weight of the compositions,
a) up to 90% by weight, preferably 20% to 90% by weight, more preferably 40% to 85% by weight, very preferably 60% to 80% by weight of a water phase,
b) up to 80% by weight, preferably 10% to 70% by weight, more preferably 20% to 60% by weight, very preferably 30% to 50% by weight of silicone oil,
c) 0.5% to 20% by weight, preferably 1% to 15% by weight, more preferably 3% to 10% by weight of one or more emulsifiers, and
d) 0.01% to 5% by weight, preferably 0.05% to 3% by weight, more preferably 0.1% to 2% by weight of copolymer A.

In a further particularly preferred embodiment the compositions of the invention are in the form of emulsions of the water-in-silicone type, preferably in the form of cosmetic or dermatological emulsions of the water-in-silicone type, and contain, based on the total weight of the compositions,
a) up to 90% by weight, preferably 20% to 90% by weight, more preferably 40% to 85% by weight, very preferably 60% to 80% by weight of a water phase,
b) up to 80% by weight, preferably 10% to 70% by weight, more preferably 20% to 60% by weight, very preferably 30% to 50% by weight of silicone oil,
c) 0.5 to 20% by weight, preferably 1% to 15% by weight, more preferably 3% to 10% by weight of one or more emulsifiers selected from the group of cetyidimethicone copolyol, lauryldimethicone copolyol, PEG/PPG-18/18 dimethicone, and trimethylsilylamodimethicone, and
d) 0.01% to 5% by weight, preferably 0.05% to 3% by weight, more preferably 0.1% to 2% by weight of copolymer A.

In a further preferred embodiment the compositions of the invention comprise one or more UV filter substances.

In a further preferred embodiment the compositions of the invention comprise one or more moisturizers and/or dyes and/or coloring pigments.

The compositions of the invention are utilized preferably for skincare.

In a further preferred embodiment the compositions of the invention are in the form of suspensions and contain, based on the total weight of the compositions
a) 0.1% to 10% by weight, preferably 0.2% to 6% by weight, more preferably 0.3% to 5% by weight of copolymer A and
b) 0.1% to 30% by weight, preferably 0.5% to 15% by weight, more preferably 1.0% to 10% by weight of solid particles, selected in particular from the group of dyes, coloring pigments, effect pigments and light-protective pigments, adsorbents and abrasive components.

In one particularly preferred embodiment the compositions of the invention are in the form of gel-based eyeshadows and contain, based on the total weight of the compositions,
a) 0.1% to 10% by weight, preferably 0.2% to 6% by weight, more preferably 0.3% to 5% by weight of copolymer A and
b) 0.1% to 30% by weight, preferably 0.5% to 15% by weight, more preferably 1.0% to 10% by weight of dyes and/or coloring pigments.

The compositions of the invention may comprise solid organic and inorganic particles. For decorative cosmetics, color pigments and colorless pigments are used. Some of the pigments specified below also serve as UV absorbers or light-protective pigments.

Particularly preferred color pigments are selected from the iron oxides having the Colour Index numbers CI 77491 (iron red), CI 77492 (iron oxide hydrate yellow) and CI 77499 (iron oxide black), from CI 77891 (titanium oxide), and carbon black. Other preferred color pigments are selected from CI 15510, CI 15585, CI 15850, CI 15985, CI 45170, CI 45370, CI 45380, CI 45425, CI 45430, CI 73360, and CI 75470. Effect pigments in the context of the present invention are pigments which by virtue of their refringency properties evoke particular optical effects. Effect pigments endow the treated surface (skin, hair, mucosa) with luster effects or glitter effects, or may scatter light diffusely and thereby optically mask unevennesses and creases in the skin. As a particular embodiment of the effect pigments, interference pigments are preferred. Examples of particularly suitable effect pigments include mica particles coated with at least one metal oxide. Besides mica, a phyllosilicate, silica gel and other SiO₂ modifications are further suitable supports. One metal oxide frequently used for coating, for example, is titanium oxide, to which iron oxide may have been admixed if desired. The reflection properties can be influenced by the size and shape (e.g., spherical, ellipsoidal, flattened, planar, nonplanar) of the pigment particles and also via the thickness of the oxide coating. Other metal oxides too, e.g., bismuth oxychloride (BiOCl), and the oxides of, for example, titanium, particularly the TiO₂ modifications anatase and rutile, and of aluminum, tantalum, niobium, zirconium, and hafnium can be used. With magnesium fluoride (MgF₂) and calcium fluoride (fluorspar, CaF₂) as well it is possible to produce effect pigments.

The effects can be controlled not only by the particle size but also via the particle size distribution of the pigment assembly. Suitable particle size distributions range, for example, from 2-50 μm, 5-25 μm, 5-40 μm, 5-60 μm, 5-95 μm, 5-100 μm, 10-60 μm, 10-100 μm, 10-125 μm, 20-100 μm, 20-150 μm, and <15 μm. A broader particle size distribution, of 20-150 μm, for example, evokes glitter effects, whereas a narrower particle size distribution of <15 μm provides a uniform satin appearance.

The compositions of the invention contain effect pigments preferably in amounts of 0.1% -20% by weight, more preferably 0.5% -10% and, with particular preference, 1% -5% by weight, based in each case on the total weight of the composition.

The preferred inorganic light-protective pigments are finely disperse or colloidally disperse metal oxides and metal salts, examples being titanium oxide, zinc oxide, iron oxide, aluminum oxide, cerium oxide, zirconium oxide, silicates (talc) and barium sulfate. The particles should in this case have an average diameter of less than 100 nm, preferably between 5 and 50 nm, and more preferably between 15 and 30 nm, referred to as nanopigments. They may have a spherical form, although it is also possible to employ particles which possess an ellipsoidal form or a form which deviates in some other way from the spherical. The pigments may also be in a surface-treated form, i.e., hydrophilized or hydrophobicized. Typical examples are coated titanium dioxides, such as titanium dioxide T 805 (Degussa) or Eusolex®T2000 (Merck). Suitable hydrophobic coating agents include, in particular, silicones, and especially trialkoxyoctylsilanes or simethicones. Particular preference is given to titanium oxide and zinc oxide.

The preferred inorganic particle substances are hydrophilic or amphiphilic. Advantageously they may have been superficially coated, particularly treated to make them superficially water-repellent. Examples thereof are titanium oxide pigments coated with aluminum stearate, zinc oxide coated with dimethylpolysiloxane (dimethicone), boron nitride coated with dimethicone, and titanium oxide coated with a mixture of dimethylpolysiloxane and silica gel and aluminum oxide hydrate, titanium oxide coated with octylsilanol, or spherical polyalkylsesquioxane particles.

Organic light-protective pigments are substances which are crystalline at room temperature and which are able to absorb ultraviolet radiation and emit the absorbed energy in the form of longer-wavelength radiation, e.g., heat. A distinction is made between UVA filters and UVB filters. The UVA and UVB filters can be used both individually and in mixtures. The organic UV filters that are suitable in accordance with the invention are selected from the derivatives, solid at room temperature, of dibenzoylmethane, cinnamic esters, diphenic esters, benzophenone, camphor, p-aminobenzoic esters, o-aminobenzoic esters, salicylic esters, benzimidazoles, 1,3,5-triazines, monomeric and oligomeric 4,4-diarylbutadienecarboxylic esters and -carboxamides, ketotricyclo[5.2.1.0]decane, benzalmalonic esters, and any desired mixtures of the components specified. The organic UV filters may be oil-soluble or water-soluble. Oil-soluble UV filters particularly preferred in accordance with the invention are 1-(4-tert-butylphenyl)-3-(4′-methoxyphenyl)propane-1,3-dione, 1-phenyl-3-(4′-isopropylphenyl)propane-1,3-dione, 3-(4′-methylbenzylidene)-D,L-camphor, 2-ethylhexyl 4-(dimethylamino)benzoate, 2-octyl 4-(dimethylamino)benzoate, amyl 4-(dimethylamino)benzoate, 2-ethylhexyl 4-methoxycinnamate, propyl 4-methoxycinnamate, isopentyl 4-methoxycinnamate, 2-ethylhexyl 2-cyano-3,3-phenylcinnamate (Octocrylene), 2-ethylhexyl salicylate, 4-isopropylbenzyl salicylate, homomethyl salicylate (3,3,5-trimethyloctylhexyl salicylate), 2-hydroxy-4-methoxybenzophenone, 2-hydroxy-4-methoxy-4′-methylbenzophenone, 2,2′-dihydroxy-4-methoxybenzophenone, di-2-ethylhexyl 4-methoxybenzmalonate, 2,4,6-trianilino(p-carbo-2′-ethyl-1′-hexyloxy)-1,3,5-triazine (Octyl Triazone) and Dioctyl Butamido Triazone, and any desired mixtures of the components specified.

Preferred water-soluble UV filters are 2-phenylbenzimidazole-5-sulfonic acid and its alkali metal, alkaline earth metal, ammonium, alkylammonium, alkanolammonium, and glucammonium salts, sulfonic acid derivatives of benzophenones, preferably 2-hydroxy-4-methoxybenzophenone-5-sulfonic acid and its salts, sulfonic acid derivatives of 3-benzylidenecamphor, such as 4-(2-oxo-3-bornylidenemethyl)benzenesulfonic acid and 2-methyl-5-(2-oxo-3-bornylidene)sulfonic acid and their salts.

In the compositions of the invention the organic and inorganic light-protective pigments are present in amounts of 0.1% -30% by weight, preferably 1% -20% by weight, and more preferably 2% to 15% by weight, based in each case on the total weight of the composition.

The compositions of the invention may comprise particulate organic or inorganic adsorbents having average particle diameters of 1-100 μm. The adsorbents are selected from pyrogenic silicas, e.g., the Aerosil grades, precipitated silicas, silica gels, silicon dioxide, clays, e.g., bentonites or kaolin, magnesium aluminum silicates, e.g., talc and boron nitride, unmodified or modified starches and starch derivatives, cellulose powders, lactoglobulin derivatives, polymer powders of polyolefins, polycarbonates, polyurethanes, polyamides, polyesters, polystyrenes, polyacrylates (meth)acrylate copolymers or (meth)acrylate-vinylidene copolymers, which may be in crosslinked form, Teflon or silicones, and also mixtures of the substances specified.

The compositions of the invention may comprise abrasive components, examples being ground plant parts such as almond bran or wheat bran, crystalline cellulose, hydrogenated jojoba oil, polymer beads, preferably of polyethylene or polyamide-11, having average diameters of 90-600 μm, and of active substance microcapsules or millicapsules, comprising petrochemical polymers (e.g., of polyamide such as nylon-11) and/or biopolymers such as gelatin, pectin, vegetable gums, alginates, and carrageenan. Preference is given to using almond bran, wheat bran, hydrogenated jojoba oil, and polyethylene beads.

In a further preferred embodiment the compositions of the invention are in the form of hair colorants, hair bleaches and/or hair tinting compositions and comprise one or more substances selected from direct dyes, oxidation dye precursors, and bleaching agents. In these compositions the copolymers A are used in the usual pH ranges, preferably as thickener and stabilizer.

Suitable direct dyes include nitroaniline derivatives, such as 1-[(2-hydroxyethyl)amino]-2-nitrobenzene (Velsol® Yellow 2), 4-hydroxypropylamino-3-nitrophenol (Velsol® Red BN), 3-nitro-p-hydroxyethylaminophenol (Velsol® Red 54), 4-hydroxyethylamino-3-nitroaniline (Velsol® Red 3), N,N′-bis(hydroxyethyl)-2-nitro-p-phenylenediamine (Velsol® Violet BS), N,N′,N′-tris(hydroxyethyl)-2-nitro-p-phenylenediamine (Velsol® Blue 2), 4-(2′-hydroxyethyl)amino-3-nitrotoluene, 4-(2′-hydroxyethyl)amino-3-nitrobenzyl alcohol, 4-(2′-hydroxyethyl)amino-3-nitro-1-trifluoromethylbenzene, 4-(2′,3′-dihydroxypropyl)amino-3-nitrochlorobenzene, 4-(2′-hydroxyethyl)amino-3-nitrobromobenzene, and 4-(2′,3′-dihydroxypropyl)amino-3-nitrobromobenzene, nitrobenzene derivatives, for example 2-amino-4-nitrophenol, picramic acid, 1-[(2′-hydroxyethyl)amino]-2-amino-4-nitrobenzene, 2-nitro-4-[(2′-hydroxyethyl)amino]aniline, 4-bis[(2′-hydroxyethyl)amino]-1-methylamino-2-nitrobenzene, 2,5-bis[(2′-hydroxyethyl)amino]nitrobenzene, 2-(2′-hydroxyethyl)amino-4,6-dinitrophenol, 1-amino-4-(2′,3′-dihydroxypropyl)amino-2-nitro-5-chlorobenzene, and also triphenylmethane dyes, such as Basic Violet 1 (C.I. 42535), azo dyes, such as Acid Brown 4 (C.I. 14805), anthraquinone dyes, such as Disperse Blue 23 (C.I. 61545), Disperse Violet 4 (C.I. 61105), 1,4,5,8-tetraaminoanthraquinone and 1,4-diaminoanthraquinone and further direct dyes.

Oxidation dye precursors available are p-phenylenediamines and p-aminophenols or derivatives thereof such as p-tolylenediamine, p-phenylenediamine, and p-aminophenol, for example, which for the purpose of shading the dyeing are combined with modifiers or couplers, such as m-phenylenediamine, resorcinol, m-aminophenol and derivatives thereof.

Suitable oxidizing agents for developing the hair dyeings, or as bleaching agents, are, preferably, hydrogen peroxide and its addition compounds. In a further preferred embodiment the composition of the invention is in the form of a hair bleach and comprises one or more oxidizing agents, preferably hydrogen peroxide.

A particular advantage is the stabilizing effect of the copolymers A on the oxidizing agents.

To increase the color intensity, the compositions of the invention may comprise the carriers which are customary in cosmetic systems, especially benzyl alcohol, vanillin (4-hydroxy-3-methoxybenzaldehyde), isovanillin, p-hydroxyanisole, 3-hydroxy-4-methoxybenzaldehyde, 2-phenoxyethanol, salicylaldehyde, 3,5-dihydroxy-benzaldehyde, 3,4-dihydroxybenzaldehyde, 4-hydroxyphenylacetamide, methyl p-hydroxybenzoate, p-hydroxybenzaldehyde, m-cresol, hydroquinone monomethyl ether, o-fluorophenol, m-fluorophenol, p-fluorophenol, 2-(2-hydroxyphenoxy)ethanol, 3,4-methylenedioxyphenol, resorcinol monomethyl ether, 3,4-dimethoxyphenol, 3-trifluoromethylphenol, resorcinol monoacetate, ethylvanillin, 2-thiopheneethanol, butyl lactate and butyl glycolate.

The hair colorants of the invention may advantageously comprise compounds which impart pearlescence, examples being fatty acid monoalkanolamides, fatty acid dialkanolamides, monoesters or diesters of alkylene glycol, particularly ethylene glycol and/or propylene glycol or its oligomers with higher fatty acids, e.g., palmitic acid, stearic acid or behenic acid or mixtures thereof, monoesters or diesters of alkylene glycols with fatty acids, fatty acids and their metal salts, monoesters or polyesters of glycerol with carboxylic acids, and keto sulfones of various kind, preferably ethylene glycol distearate and polyethylene glycol distearate with about 3 glycol units.

In one further preferred embodiment the compositions of the invention are in the form of deodorants or antiperspirants. In this case the copolymers A are used, preferably, as a thickener, consistency agent, emulsifier, solubilizer, dispersant, lubricant, adhesive, conditioners, and stabilizer.

The deodorants and antiperspirants of the invention contain, based on the finished compositions, preferably 0.01% to 10% by weight, more preferably 0.1% to 5% by weight, very preferably 0.5% to 3% by weight of the copolymers A.

In a further preferred embodiment the compositions of the invention contain 0.01% to 89% by weight, more preferably 5% to 50% by weight, very preferably 10% to 30% by weight of water.

In a further preferred embodiment the compositions of the invention contain up to 10% by weight, preferably 1% to 6% by weight, of glycerol.

In a further preferred embodiment the compositions of the invention comprise active antimicrobial substances which inhibit the microorganisms which break down perspiration or the esterase enzyme which breaks down perspiration.

Of preferential suitability as active antimicrobial substances are cetyltrimethylammonium chloride, cetylpyridinium chloride, benzethonium chloride, diisobutylethoxyethyldimethylbenzylammonium chloride, sodium N-laurylsarcosinate, sodium N-palmethylsarcosinate, lauroylsarcosine, N-myristoylglycine, potassium N-laurylsarcosine, trimethylammonium chloride, sodium aluminum chlorohydroxylactate, triethyl citrate, tricetylmethylammonium chloride, 2,4,4′-trichloro-2′-hydroxydiphenyl ether (Triclosan), phenoxyethanol, 1,5-pentanediol, 1,6-hexanediol, 3,4,4′-trichlorocarbanilide (Triclocarban), diaminoalkylamide, for example L-lysinehexadecylamide, heavy metal citrate salts, salicylates, piroctose, especially zinc salts, pyrithiones and their heavy metal salts, especially zinc pyrithione, zinc phenolsulfate, farnesol and combinations of these active compounds.

The compositions of the invention contain the antimicrobial agents preferably in amounts up to 50% by weight, more preferably 0.01% to 10% by weight, very preferably 0.1% to 10% by weight.

In a further preferred embodiment the compositions of the invention comprise astringents.

Preferred astringents are oxides, preferably magnesium oxide, aluminum oxide, titanium dioxide, zirconium dioxide, and zinc oxide, oxide hydrates, preferably aluminum oxide hydrate (boehmite), and hydroxides, preferably of calcium, magnesium, aluminum, titanium, zirconium or zinc.

The compositions of the invention contain the active astringent substances in amounts preferably of 0 to 50% by weight, more preferably 0.01% to 10% by weight, and very preferably 0.1% to 10% by weight.

In a further preferred embodiment the compositions of the invention comprise gelling agents.

Suitable gelling agents are all surface-active substances which, in solution in the liquid phase, form a network structure and so solidify the liquid phase. Suitable gelling agents are specified for example in WO 98/58625.

Preferred gelling agents are metal salts of fatty acids, preferably with 12 to 22 carbon atoms, examples being sodium stearate, sodium palmitate, sodium laurate, sodium arachidates, sodium behenate, potassium stearate, potassium palmitate, sodium myristate, aluminum monostearate, hydroxy fatty acids, examples being 12-hydroxystearic acid, 12-hydroxylauric acid, and 16-hydroxyhexadecanoic acid; fatty acid amides; fatty acid alkanolamides; dibenzalsorbitol, and alcohol-soluble polyamides and polyacrylamides or mixtures of such.

The compositions of the invention preferably contain 0.01% to 20% by weight, more preferably 0.1% to 10% by weight, with particular preference 1% to 8% by weight, and with very particular preference 3% to 7% by weight of gelling agents.

The copolymers A are outstanding thickeners for aqueous, aqueous-alcoholic or aqueous-surfactant formulations and endow the compositions with a more transparent appearance than with Aristoflex®AVC.

The amount of copolymers A used in compositions on a purely aqueous basis, an aqueous-alcoholic basis or an aqueous-surfactant basis is preferably in the range from 0.01% to 10% by weight, more preferably 0.1% to 5% by weight, very preferably 0.5% to 3% by weight.

The copolymers A have diverse possibilities for use and are suitable, for example for use in aqueous, aqueous-alcoholic, and aqueous-surfactant formulations, emulsions, suspensions, dispersions, and powders. In one preferred embodiment the copolymers A are used in rinse-off products, preferably shampoos, shower products, including shower gels, and foam-bath products.

In another preferred embodiment the copolymers A are used in leave-on products, preferably skincare compositions, day creams, night creams, beauty creams, nutrient creams, body lotions, ointments, sun protection products, lip care products, and deodorants.

In another preferred embodiment the compositions of the invention are in the form of surfactant-free, aqueous compositions and emulsions: for example, as haircare products, hair treatments and hair rinses, hair gels, hair styling products, but also as perming products, hair colorants, and also as decorative cosmetics, examples being makeups, eyeshadows, lipsticks, mascaras, and the like. In one particularly preferred embodiment the compositions of the invention are in the form of hair colorants, hair bleaches, haircare or hair styling compositions.

The copolymers A used in accordance with the invention are suitable for producing pumpable, sprayable and/or foamable non-aerosol products. In one preferred embodiment, therefore, the compositions of the invention are in a pumpable, sprayable and/or foamable form. These compositions preferably contain no aerosol.

In one particularly preferred embodiment the copolymers A are incorporated into sprayable, pumpable, and foamable gels and foams, especially in sprayable hair gels and foamable sun protection products, and bring about an improvement in the spray characteristics of the compositions, with optimized droplet size distribution.

Advantage attaches to a hair gel composition comprising at least one copolymer A and at least one hairsetting polymer.

The viscosity of the gels is preferably 100 to 5000 mPa*s, more preferably 200 to 1000 mPa*s, very preferably 250 to 800 mPa*s, measured as the dynamic viscosity using a Bohlin Rheometer CS, measuring element C25, at a temperature of 25° C. and a shear rate of 50 s⁻¹.

The gel-forming copolymer A is used preferably in an amount of 0.1% to 10%, more preferably of 0.2% to 8% by weight, and the hairsetting polymer in an amount of preferably 0.1% to 15%, more preferably of 0.5% to 10% by weight.

The hairsetting polymer may be nonionic, anionic, cationic or amphoteric, but is preferably nonionic or anionic. It may be a synthetic or a natural polymer. Natural polymers are taken to include chemically modified polymers of natural origin. Preferred polymers in particular are those which possess sufficient solubility in water, alcohol or water/alcohol mixtures to be present in fully dissolved form in the composition of the invention. By hairsetting polymers are meant those polymers which on application to the hair as a 0.01% to 5% aqueous, alcoholic or aqueous-alcoholic solution or dispersion are capable of producing a hairsetting effect.

Suitable synthetic, nonionic hairsetting polymers are homopolymers or copolymers constructed from at least one of the following monomers: vinylpyrrolidone, vinylimidazole, vinylcaprolactam, vinyl esters such as vinyl acetate, vinyl alcohol, acrylamide, methacrylamide, alkyl- and dialkylacrylamide, alkyl- and dialkylmethacrylamide, dialkylaminoalkylmethacrylamide, dialkylaminoalkylacrylamide, alkyl acrylate, alkyl methacrylate, propylene glycol or ethylene glycol, the alkyl groups of these monomers being preferably C₁ to C₇ alkyl groups, more preferably C₁ to C₃ alkyl groups. Suitable examples include homopolymers of vinylcaprolactam, of vinylpyrrolidone or of N-vinylformamide. Examples of further suitable hairsetting polymers are copolymers of vinylpyrrolidone and vinyl acetate, terpolymers of vinylpyrrolidone, vinyl acetate, and vinyl propionate, terpolymers of vinylpyrrolidone, vinylcaprolactam, and dialkylaminoalkyl (meth)acrylate, terpolymers of vinylpyrrolidone, vinylcaprolactam, and dialkylaminoalkyl (meth)acrylamide, terpolymers of vinylpyrrolidone, vinylimidazole, and (meth)acrylamide, polyacrylamide, polyvinyl alcohol, and also hairsetting polyethylene glycol/polypropylene glycol copolymers. Particularly preferred nonionic polymers are polyvinylpyrrolidone and polyvinylpyrrolidone/vinyl acetate copolymers. Preference is given to nonionic vinyllactam homopolymers and copolymers. Examples of suitable vinyllactams include vinylcaprolactam and vinylpyrrolidone. Particular preference is given to polyvinylpyrrolidone, polyvinylcaprolactam, terpolymers of vinylpyrrolidone, vinylimidazole, and (meth)acrylamide, and vinylpyrrolidone/vinyl acetate copolymers. Preferred commercial products are Luviskol® VA 37, Luviskol® VA 64 and Luviset® Clear.

Suitable anionic hairsetting polymers may be natural or synthetic homopolymers or copolymers with monomer units containing acid groups, copolymerized if appropriate with comonomers containing no acid groups. The acid groups are preferably selected from —COOH, —SO₃H, —OSO₃H, —OPO₂H and —OPO₃H₂, among which the carboxylic acid groups are preferred. The acid groups may be in unneutralized form or in partly or fully neutralized form. They are preferably present from 50% to 100% in anionic or neutralized form. Neutralizing agents which can be used are those specified above. Suitable monomers are unsaturated, free-radically polymerizable compounds which carry at least one acid group, especially carboxyvinyl monomers. Suitable monomers containing acid groups are, for example, acrylic acid, methacrylic acid, crotonic acid, maleic acid or maleic anhydride or their monoesters, aldehydocarboxylic acids or ketocarboxylic acids.

Examples of comonomers not substituted by sulfo groups are acrylamide, methacrylamide, alkyl- and dialkylacrylamide, alkyl- and dialkylmethacrylamide, alkyl acrylate, alkyl methacrylate, vinylcaprolactone, vinylpyrrolidone, vinyl esters, vinyl alcohol, propylene glycol or ethylene glycol, amine-substituted vinyl monomers such as dialkylaminoalkyl acrylate, dialkylaminoalkyl methacrylate, monoalkylaminoalkyl acrylate and monoalkylaminoalkyl methacrylate, the alkyl groups of these monomers being preferably C₁ to C₇ alkyl groups, more preferably C₁ to C₃ alkyl groups.

Suitable anionic polymers are, in particular, copolymers of acrylic acid or methacrylic acid with monomers selected from acrylic or methacrylic esters, acrylamides, methacrylamides, and vinylpyrrolidone, homopolymers of crotonic acid, and copolymers of crotonic acid with monomers selected from vinyl esters, acrylic or methacrylic esters, acrylamides, and methacrylamides. An example of a suitable natural polymer is shellac.

Preferred anionic polymers are crosslinked or noncrosslinked vinyl acetate/crotonic acid copolymers. Preference is likewise given to partially esterified copolymers of vinyl methyl ether with maleic anhydride. Further suitable anionic polymers are, for example, terpolymers of acrylic acid, alkyl acrylate, and N-alkylacrylamide, especially acrylic acid/ethyl acrylate/N-t-butylacrylamide terpolymers, or terpolymers of vinyl acetate, crotonate, and vinyl alkanoate, especially vinyl acetate/crotonate/vinyl neodecanoate copolymers.

Suitable film-forming amphoteric polymers are polymers which in addition to acidic or anionic groups contain, as further functional groups, basic or cationic groups, especially primary, secondary, tertiary or quaternary amine groups. Examples of such copolymers formed from alkylacrylamide, alkylaminoalkyl methacrylate, and two or more monomers selected from acrylic acid, methacrylic acid or their esters, the alkyl groups containing 1 to 4 carbon atoms and at least one of the monomers containing an acid group.

Further examples of suitable hairsetting polymers are copolymers of acrylic acid, methacrylate, and methacrylamidopropyltrimethylammonium chloride, copolymers of acrylamidopropyltrimethylammonium chloride and acrylates, copolymers of acrylamide, acrylamidopropyltrimethylammonium chloride, 2-amidopropylacrylamide sulfonate, and dimethylaminopropylamine or chitosans. Also suitable are polymers with monomers which carry betaine groups, such as copolymers of methacryloylethylbetaine and two or more monomers of acrylic acid or its simple esters, known under the INCI name Methacryloyl Ethyl Betaine/Acrylate Copolymer.

In one preferred embodiment the composition of the invention is formulated in an aqueous medium, an alcoholic medium or an aqueous-alcoholic medium containing preferably at least 10% by weight, more preferably at least 50% by weight, of water and, preferably, not more than 40% by weight of alcohol.

Alcohols present may in particular be the lower monoalcohols having 1 to 4 carbon atoms that are commonly used for cosmetic purposes, such as ethanol and isopropanol.

The hair gels of the invention are clear, transparent or translucent, colorless compositions having particularly positive spraying properties.

The cosmetic and pharmaceutical compositions of the invention may comprise anionic, cationic, nonionic, zwitterionic and/or amphoteric surfactants.

The total amount of surfactants used in the compositions of the invention (in the case of rinse-off products, for example), based on the total weight of the compositions, is preferably 2% to 70% by weight, more preferably 5% to 40% by weight, very preferably 12% to 35% by weight.

Preferred anionic surfactants are (C₁₀-C₂₀) alkyl- and alkylene-carboxylates, alkyl ether carboxylates, fatty alcohol sulfates, fatty alcohol ether sulfates, alkylamide sulfates and alkylamidesulfonates, fatty acid alkylamide polyglycol ether sulfates, alkanesulfonates and hydroxyalkanesulfonates, olefinsulfonates, acyl esters of isethionates, α-sulfo fatty acid esters, alkylbenzenesulfonates, alkylphenol glycol ether sulfonates, sulfosuccinates, sulfosuccinic monoesters and diesters, fatty alcohol ether phosphates, protein-fatty acid condensates, alkylmonoglyceride sulfates and alkylmonoglyceride sulfonates, alkylglyceride ether sulfonates, fatty acid methyltaurides, fatty acid sarcosinates, sulforicinoleates, and acylglutamates. These compounds and mixtures thereof are used in the form of their water-soluble or water-dispersible salts, examples being the sodium, potassium, magnesium, ammonium, mono-, di-, and triethanolammonium salts and also analogous alkylammonium salts.

The weight fraction of the anionic surfactants, based on the total weight of the compositions, is preferably 2% to 30% by weight, more preferably 5% to 25% by weight, very preferably 12% to 22% by weight.

Preferred cationic surfactants are quaternary ammonium salts, such as di-(C₁₀-C₂₄)-alkyl-dimethylammonium chloride or bromide, preferably di-(C₁₂-C₁₈)-alkyl-dimethylammonium chloride or bromide; (C₁₀-C₂₄)-alkyldimethyl-ethylammonium chloride or bromide; (C₁₀-C₂₄)-alkyl-trimethylammonium chloride or bromide, preferably cetyltrimethylammonium chloride or bromide and (C₂₀-C₂₂)-alkyl-trimethylammonium chloride or bromide; (C₁₀-C₂₄)-alkyl-dimethylbenzylammonium chloride or bromide, preferably (C₁₂-C₁₈)-alkyl-dimethylbenzylammonium chloride; N—(C₁₀-C₁₈)-alkyl-pyridinium chloride or bromide, preferably N—(C₁₂-C₁₆)-alkyl-pyridinium chloride or bromide; N—(C₁₀-C₁₈)-alkyl-isoquinolinium chloride, bromide or -monoalkyl sulfate; N—(C₁₂-C₁₈)-alkyl-polyoylaminoformylmethylpyridinium chloride; N—(C₁₂-C₁₈)-alkyl-N-methylmorpholinium chloride, bromide or monoalkyl sulfate; N—(C₁₂-C₁₈)-alkyl-N-ethylmorpholinium chloride, bromide or monoalkyl sulfate; (C₁₆-C₁₈)-alkyl-pentaoxethylammonium chloride; diisobutylphenoxyethoxyethyldimethylbenzylammonium chloride; salts of N,N-diethylaminoethylstearylamide and oleylamide with hydrochloric acid, acetic acid, lactic acid, citric acid, phosphoric acid; N-acylaminoethyl-N,N-diethyl-N-methylammonium chloride, bromide or monoalkyl sulfate and N-acylaminoethyl-N,N-diethyl-N-benzylammonium chloride, bromide or monoalkyl sulfate, acyl being preferably stearyl or oleyl.

The weight fraction of the cationic surfactants, based on the total weight of the compositions, is preferably 1% to 10% by weight, more preferably 2% to 7% by weight, with particular preference 3% to 5% by weight.

Preferred nonionic surfactants are fatty alcohol ethoxylates (alkylpolyethylene glycols); alkylphenolpolyethylene glycols; alkyl mercaptan polyethylene glycols; fatty amine ethoxylates (alkylaminopolyethylene glycols); fatty acid ethoxylates (acylpolyethylene glycols); polypropylene glycol ethoxylates (Pluronics®); fatty acid alkylolamides (fatty acid amide polyethylene glycols); N-alkyl-, N-alkoxypolyhydroxy fatty acid amide, sucrose esters; sorbitol esters, and the polyglycol ether.

The weight fraction of the nonionic surfactants in the compositions of the invention (in the case of rinse-off products, for example), based on the total weight of the compositions, is preferably in the range from 1% to 20% by weight, more preferably from 2% to 10%, and with particular preference from 3% to 7% by weight.

Preferred amphoteric surfactants are N—(C₁₂-C₁₈)-alkyl-p-aminopropionates and N—(C₁₂-C₁₈)alkyl-p-iminodipropionates in the form of the alkali metal and mono-, di-, and trialkylammonium salts; N-acylaminoalkyl-N,N-dimethylacetobetaine, preferably N—(C₈-C₁₈)-acylaminopropyl-N,N-dimethylacetobetaine; (C₁₂-C₁₈)-alkyl-dimethylsulfopropylbetaine; amphoteric surfactants based on imidazoline (trade name: Miranol®, Steinapon®), preferably the sodium salt of 1-(β-carboxymethyloxyethyl)-1-(carboxymethyl)-2-laurylimidazolinium; amine oxide, e.g., (C₁₂-C₁₈)-alkyl-dimethylamine oxide, and fatty acid amidoalkyl-dimethylamine oxide.

The weight fraction of the amphoteric surfactants, based on the total weight of the compositions, is preferably 0.5% to 20% by weight and more preferably 1% to 10% by weight.

Preferred surfactants are lauryl sulfate, laureth sulfate, cocoamidopropylbetaine, sodium cocoylglutamate, and lauroamphoacetate.

In one preferred embodiment the compositions additionally comprise, as foam enhancers, co-surfactants from the group of alkylbetaines, alkylamidobetaines, aminopropionates, aminoglycinates, imidazolinium betaines and sulfobetaines, amine oxides, fatty acid alkanolamides, and polyhydroxyamides.

The compositions of the invention may comprise, as further auxiliaries and additives, adjuvants which are customary within cosmetology, such as, for example, cationic polymers, film formers, superfatting agents, stabilizers, active biogenic substances, glycerol, preservatives, pearlescents, colorants, fragrances, solvents, opacifiers, and also protein derivatives such as gelatin, collagen-hydrolysates, natural and synthetic polypeptides, egg yolk, lecithin, lanolin and lanolin derivatives, fatty alcohols, silicones, deodorants, substances having keratolytic and keratoplastic action, enzymes, and carrier substances. Additionally it is possible to add antimicrobial agents to the compositions of the invention.

Suitable cationic polymers are those known under the INCI name “Polyquaternium”, particularly Polyquaternium-31, Polyquaternium-16, Polyquaternium-24, Polyquaternium-7, Polyquaternium-22, Polyquatemium-39, Polyquaternium-28, Polyquatemium-2, Polyquaternium-10, Polyquaternium-11, and also Polyquaternium 37&mineral oil&PPG trideceth (Salcare SC95), PVP-dimethylaminoethyl methacrylate copolymer, guar-hydroxypropyltriammonium chlorides, and also calcium alginate and ammonium alginate. Use may be made additionally of cationic cellulose derivatives; cationic starch; copolymers of diallylammonium salts and acrylamides; quatemized vinylpyrrolidone/vinylimidazole polymers; condensates of polyglycols and amines; quaternized collagen polypeptides; quaternized wheat polypeptides; polyethyleneimines; cationic silicone polymers, such as amidomethicone; copolymers of adipic acid and dimethylaminohydroxypropyidiethylenetriamine; polyaminopolyamide and cationic chitin derivatives, such as chitosan. Examples of suitable silicone compounds include dimethylpolysiloxane, methylphenylpolysiloxanes, cyclic silicones, and amino-, fatty acid-, alcohol-, polyether-, epoxy-, fluoro- and/or alkyl-modified silicone compounds, and also polyalkylsiloxanes, polyalkylarylsiloxanes, polyethersiloxane copolymers, as described in US 5,104,645 and the specifications cited therein, which can be present at room temperature either in liquid form or in resinous form.

Suitable film formers, depending on the intended application, are water-soluble polyurethanes, examples being C₁₀ polycarbamyl polyglyceryl esters, polyvinyl alcohol, polyvinylpyrrolidone, copolymers thereof, such as vinylpyrrolidone/vinyl acetate copolymer, water-soluble acrylic acid polymer/copolymers and their esters or salts, examples being partial ester copolymers of acrylic/methacrylic acid and polyethylene glycol ethers of fatty alcohols, such as acrylate/steareth-20 methacrylate copolymer, water-soluble cellulose, such as hydroxymethylcellulose, hydroxyethylcellulose, hydroxypropylcellulose, water-soluble quaterniums, polyquaterniums, carboxyvinyl polymers, such as carbomers and their salts, and polysaccharides, such as polydextrose and glucan.

Superfatting agents which can be used are substances such as, for example, polyethoxylated lanolin derivatives, lecithin derivatives, polyol fatty acid esters, monoglycerides, and fatty acid alkanolamides, the latter serving simultaneously as foam stabilizers. Moisturizing substances available are, for example, isopropyl palmitate, glycerol and/or sorbitol.

As stabilizers it is possible to use metal salts of fatty acids, such as magnesium stearate, aluminum stearate and/or zinc stearate.

By active biogenic substances are meant, for example, plant extracts and vitamin complexes.

The compositions of the invention may further comprise organic solvents. Suitable organic solvents in principle include all monohydric or polyhydric alcohols. Preference is given to using alcohols having 1 to 4 carbon atoms such as ethanol, propanol, isopropanol, n-butanol, isobutanol, tert-butanol, glycerol, and mixtures of said alcohols. Further preferred alcohols are polyethylene glycols having a relative molecular mass of below 2000. Particular preference is given to using polyethylene glycol having a relative molecular mass of between 200 and 600 in amounts of up to 45% by weight, and polyethylene glycol having a relative molecular mass of between 400 and 600 in amounts of 5% to 25% by weight. Examples of further suitable solvents include triacetin (glyceryl triacetate) and 1-methoxy-2-propanol.

The compositions of the invention can be blended with conventional ceramides, pseudoceramides, fatty acid N-alkylpolyhydroxyalkylamides, cholesterol, cholesterol fatty acid esters, fatty acids, triglycerides, cerebrosides, phospholipids, and similar substances as a care addition.

Examples of suitable UV filters include 4-aminobenzoic acid; 3-(4′-trimethylammonium)benzylideneboran-2-one methyl sulfate; 3,3,5-trimethylcyclohexyl salicylate; 2-hydroxy-4-methoxybenzophenone; 2-phenylbenzimidazole-5-sulfonic acid and its potassium, sodium, and triethanolamine salts; 3,3′-(1,4-phenylenedimethine)bis(7,7-dimethyl-2-oxobicyclo[2.2.1]heptane-1 -methanesulfonic acid and its salts; 1-(4-tert-butylphenyl)-3-(4-methoxyphenyl)propane-1,3-dione, 3-(4′-sulfo)benzylidenebornan-2-one and its salts; 2-ethylhexyl 2-cyano-3,3-diphenylacrylate; polymer of N-[2(and 4)-(2-oxoborn-3-ylidenemethyl)benzyl]acrylamide; 2-ethylhexyl 4-methoxycinnamate; ethoxylated ethyl-4-aminobenzoate; isoamyl 4-methoxycinnamate; 2,4,6-tris[p-(2-ethylhexyloxycarbonyl)anilino]-1,3,5-triazine; 2-(2H-benzotriazol-2-yl)-4-methyl-6-(2-methyl-3-(1,3,3,3-tetramethyl-1-(trimethylsilyloxy)disiloxanyl)propyl)phenol; 4,4′-[(6-[4-((1,1-dimethylethyl)aminocarbonyl)phenylamino]-1,3,5-triazin-2,4-yl)diimino]bis(2-ethylhexyl benzoate); 3-(4′-methylbenzylidene)-D,L-camphor; 3-benzylidenecamphor; 2-ethylhexylsalicylate; 2-ethylhexyl 4-dimethylaminobenzoate; hydroxy-4-methoxybenzophenone-5-sulfonic acid (sulisobenzonum) and the sodium salt; and/or 4-isopropylbenzyl salicylate.

Advantageous compositions of the invention comprise one or more antioxidants.

As antioxidants which are favorable, and yet whose use is optional, it is possible to use all antioxidants that are customary or suitable for cosmetic, dermatological and/or pharmaceutical applications.

Advantageously the antioxidants are selected from the group consisting of amino acids (e.g., glycine, histidine, tyrosine, tryptophan) and their derivatives, imidazoles (e.g., urocaninic acid) and their derivatives, peptides such as D,L-carnosine, D-carnosine, L-camosine and their derivatives (e.g., anserine), carotenoids, carotenes (e.g., α-carotene, β-carotene, lycopene) and their derivatives, chlorogenic acid and its derivatives, lipoic acid and its derivatives (e.g., dihydrolipoic acid), aurothioglucose, propylthiouracil and other thiols (e.g., thioredoxin, glutathione, cysteine, cystine, cystamine and their glycosyl, N-acetyl, methyl, ethyl, propyl, amyl, butyl and lauryl, palmitoyl, oleyl, y-linoleyl, cholesteryl, and glyceryl esters) and also their salts, dilauryl thiodipropionate, distearyl thiodipropionate, thiodipropionic acid and derivatives thereof (esters, ethers, peptides, lipids, nucleotides, nucleosides, and salts) and also sulfoximine compounds (e.g., buthionine sulfoximine, homocysteine sulfoximine, buthionine sulfone, penta-, hexa-, and heptathionine sulfoximine) in very low tolerable doses (e.g., pmol to pmol/kg), and also (metal) chelators (e.g., α-hydroxy fatty acids, palmitic acid, phytic acid, lactoferrin), a-hydroxy acids (e.g., citric acid, lactic acid, malic acid), humic acid, bile acid, bile extracts, bilirubin, biliverdin, EDTA, EGTA and their derivatives, unsaturated fatty acids and their derivatives (e.g., γ-linolenic acid, linoleic acid, oleic acid), folic acid and its derivatives, ubiquinone and ubiquinol and their derivatives, vitamin C and derivatives (e.g., ascorbyl palmitate, Mg ascorbyl phosphate, ascorbyl acetate), tocopherols and derivatives (e.g., vitamin E acetate), vitamin A and derivatives (vitamin A palmitate), and also coniferyl benzoate of benzoin resin, rutic acid and its derivatives, α-glycosylrutin, ferulic acid, furfurylideneglucitol, carnosine, butylated hydroxytoluene, butylated hydroxyanisole, nordihydroguaiaretic acid, nordihydroguaiac resin acid, nordihydroguaiaretic acid, trihydroxybutyrophenone, uric acid and its derivatives, mannose and its derivatives, zinc and its derivatives (e.g., ZnO, ZnSO₄), selenium and its derivatives (e.g., selenomethionine), stilbenes and their derivatives (e.g., stilbene oxide, trans-stilbene oxide), superoxide dismutase, and the derivatives suitable in accordance with the invention (salts, esters, ethers, sugars, nucleotides, nucleosides, peptides, and lipids) of these stated substances.

With particular advantage for the purposes of the present invention it is possible to use water-soluble antioxidants.

The antioxidants can protect the skin and the hair against oxidative stress. Preferred antioxidants here are vitamin E and its derivatives and also vitamin A and its derivatives.

The amount of the antioxidants (one or more compounds) in the compositions of the invention is preferably 0.001% to 30% by weight, more preferably 0.05% to 20% by weight, in particular 1% to 10% by weight, based on the total weight of the compositions.

Where vitamin E and/or its derivatives constitute the antioxidant or antioxidants, it is advantageous to select their respective concentrations from the range from 0.001% to 10% by weight, based on the total weight of the compositions.

Where vitamin A, or vitamin A derivatives, or carotenes or their derivatives constitute the antioxidant or antioxidants, it is advantageous to select their respective concentrations from the range from 0.001% to 10% by weight, based on the total weight of the compositions.

In one particularly preferred embodiment of the invention the cosmetic or pharmaceutical compositions comprise antioxidants selected from superoxide dismutase, tocopherol (vitamin E) and ascorbic acid (vitamin C).

Examples of suitable preservatives include phenoxyethanol, parabens, pentanediol or sorbic acid.

Colorants which can be used are the substances which are approved and suitable for cosmetics use.

Suitable active antifungal substances include, preferably, ketoconazole, oxiconazole, bifonazole, butoconazole, cloconazole, clotrimazole, econazole, enilconazole, fenticonazole, isoconazole, miconazole, sulconazole, tioconazole, fluconazole, itraconazole, terconazole, naftifins and terbinafine, Zn pyrithione and octopirox.

In order to adjust the rheological properties of aqueous or solvent-containing emulsions or suspensions, a multiplicity of different systems are reported in the technical literature. Known examples include cellulose ethers and other cellulose derivatives (e.g., carboxymethylcellulose, hydroxyethylcellulose), gelatin, starch and starch derivatives, sodium alginates, fatty acid polyethylene glycol esters, agar agar, tragacanth or dextrins. As synthetic polymers a variety of materials are employed, such as polyvinyl alcohols, polyacrylamides, polyvinylamides, polysulfonic acids, polyacrylic acid, polyacrylic esters, polyvinylpyrrolidone, polyvinyl methyl ether, polyethylene oxides, copolymers of maleic anhydride and vinyl methyl ether, and also diverse mixtures and copolymers of the aforementioned compounds, including their various salts and esters. These polymers may alternatively be in crosslinked form or uncrosslinked.

It is important for the invention that the copolymers A can be employed even without the use of an additional coemulsifier and/or without the use of an additional consistency agent. The use of coemulsifiers and/or consistency agents, therefore, though possible, is not mandatory. A combination with other known coemulsifiers and/or consistency agents may be desirable in order to set specific cosmetic profiles and to exploit synergistic effects.

The compositions of the invention are usually adjusted to a pH in the range 2 to 12, preferably 3 to 8.

The nature of the compositions of the invention is extremely advantageous: the emulsions are creamy and ointmentlike and do not at all have the gellike or even gelatinlike appearance of certain prior art emulsions in which the external aqueous phase has been thickened.

The cosmetic sensation on the skin as well is very good. On application, the emulsion imparts a sensation of freshness and of comfort, and at the same time has a rich, nourishing effect; it is soft and comfortable and in no way sticky.

The compositions of the invention can be formulated without aerosol in a readily sprayable form.

The examples and applications below are intended to illustrate the invention, though without restricting it to them (all percentages are weight %).

### EXAMPLES

Preparation of Copolymers

### Example A

A 1000-ml flask with anchor stirrer, reflux condenser, internal thermometer, introduction facility for N₂ and NH₃ was charged with 501 g of tert-butanol and 14 g of water. Subsequently 80.00 g of 2-acrylamido-2-methylpropanesulfonic acid were introduced and dispersed with vigorous stirring, the solvent retaining its turbidity. Over a period of 30 minutes 6.64 g of ammonia were introduced into the gas space above the dispersion, which was stirred for at least 30 minutes more until a pH of 6 to 7 was established. 4.10 g of N-vinylcaprolactam and 0.8 g of allyl methacrylate were added and the reservoir vessel was rinsed out in each case with tert-butanol (about 6 ml) in order to minimize losses during the addition. The reaction mixture was then heated to a temperature of 60° C., in the course of which it was rendered inert by simultaneous introduction of N₂. When the temperature of 60° C. had been reached, 1.0 g of dilauroyl peroxide was added. Onset of reaction occurred immediately following the addition of the initiator, as was evident from a rise in the temperature and from the flocculation of the polymer. About 15 minutes after the onset of the polymerization reaction the nitrogen feed was shut off. Approximately 30 minutes after the dilauroyl peroxide initiator had been added, the temperature reached a maximum (about 65-70° C.). A further 30 minutes after this maximum had been passed, the mixture was heated to reflux and subsequently stirred under these conditions for two hours. In the course of the reaction the contents of the reaction vessel took on a porridgy consistency, but remained readily stirrable. Subsequently the mixture was cooled to room temperature and the solid was filtered off with suction. The paste was dried in a vacuum drying oven at 60-70° C. over 24 hours. This gave 92.2 g of a fine white powder.

### Example B

In accordance with Example A, the crosslinked copolymer was prepared from 55 g of 2-acrylamido-2-methylpropanesulfonic acid, 35 g of N-vinylcaprolactam, and 1.9 g of trimethylolpropane trimethacrylate.

### Example C

In accordance with Example A, the crosslinked copolymer was prepared from 80 g of 2-acrylamido-2-methylpropanesulfonic acid, 4.2 g of N-vinylcaprolactam, and 1.8 g of trimethylolpropane triacrylate.

Skincare Compositions

In application examples 1 to 25 below, each of the copolymers from examples A, B, and C were used. These examples are denoted correspondingly as example 1A (containing the copolymer from example A), 1B (containing the copolymer from example B), 1C (containing the copolymer from example C), 2A (containing the copolymer from example A), and so on.

### Examples 1A, 1B and 1C

### Skin Milk, Sprayable

| | | | |
|---|---|---|---|
| A | Hostaphat ® KL 340 D | (Clariant) | 1.00% |
| | Trilaureth-4 phosphate | | |
| | Mineral oil, low-viscosity | | 8.00% |
| | Isopropyl palmitate | | 3.00% |
| | Cetearyl alcohol | | 0.50% |
| | Myritol ® 318 | | 2.00% |
| | Caprylic/capric triglycerides | | |
| | Tegin ® M | | 0.50% |
| | Glyceryl stearate | | |
| | SilCare ® Silicone 41M15 | (Clariant) | 1.00% |
| | Caprylyl methicone | | |
| B | Copolymer from example A, B or C | | 0.40% |
| C | Water | | ad 100.00% |
| | Glycerol | | 5.00% |
| D | Fragrance | | 0.30% |
| | Alcohol | | 5.00% |
| | Tocopheryl acetate | | 1.00% |
| E | Nipaguard ® PDU | (Clariant) | q.s. |
| | Propylene glycol (and) diazolidinylurea | | |
| | (and) methylparaben (and) propylparaben | | |

Preparation:

I Melt A at 60° C., then add B

II Heat C to 60° C.

III Stir II into I, stir until cooling to room temperature

IV Add components D successively to III at 35° C.

V Add E and homogenize the emulsion

### Examples 2A, 2B and 2C

### Antiacne Gel

| | | | |
|---|---|---|---|
| A | Octopirox ® | (Clariant) | 0.10% |
| | Piroctone olamine | | |
| B | Ethanol | | 25.00% |
| | Propylene glycol | | 20.00% |
| C | Perfume | | 0.20% |
| | Nipaguard ® DCB | (Clariant) | q.s. |
| | Phenoxyethanol (and) methyldibromo | | |
| | glutaronitrile | | |
| D | Copolymer from Example A, B or C | | 1.30% |
| E | Allantoin | (Clariant) | 0.10% |
| | Allantoin | | |
| F | Water | | ad 100% |

Preparation:

I Dissolve A in B

II Add C to I

III Stir D into II

IV Dissolve E in F at 35° C.

V Add IV to III with stirring

### Examples 3A, 3B and 3C

### Moisturizer

| | | | |
|---|---|---|---|
| A | Water | | ad 100% |
| B | Glycerol | | 5.00% |
| | Bozequat ® 4000 | (Clariant) | 0.60% |
| | Polyquaternium-43 | | |
| | Polyglycol 35000 S | (Clariant) | 0.50% |
| | PEG-800 | | |
| | Copolymer from Example A, B or C | | 0.60% |
| | Fragrance | | 0.15% |
| | Nipaguard ® MPA | (Clariant) | q.s. |
| | Benzyl alcohol (and) methylparaben (and) | | |
| | propylparaben | | |

Preparation:

Stir components B successively into A.

### Examples 4A, 4B and 4C

### Face Gel

| | | | |
|---|---|---|---|
| A | Copolymer from Example A, B or C | | 1.50% |
| B | Water | | ad 100% |
| C | Glycerol | | 4.00% |
| | Propylene glycol | | 3.00% |
| | Urea | | 0.50% |
| | Panthenol | | 0.30% |
| | Sorbitol | | 0.50% |
| D | Biobranil, water-soluble | | 1.00% |
| | Water (aqua), propylene glycol, wheat bran | | |
| | extract, ethoxydiglycol, PEG-40 | | |
| | hydrogenated castor oil, trideceth-9, | | |
| | tocopherol | | |
| | Aloe vera gel concentrate 10:1 | | 1.00% |
| | Water (aqua), aloe barbadensis gel | | |
| | Hamamelis: | | 1.00% |
| | Ethoxydiglycol, propylene glycol, water | | |
| | (aqua), butylene glycol, witch hazel | | |
| | (Hamamelis virginiana) extract | | |
| | Mango | | 1.00% |
| | Water (aqua), ethoxydiglycol, propylene | | |
| | glycol, mango (Mangifera indica) extract, | | |
| | butylene glycol | | |
| | Hyaluronic acid | | 0.50% |
| | Hydroviton 24 | | 2.00% |
| | Water (aqua), sodium lactate, lactic acid, | | |
| | glycerol, serine, sorbitol, EA lactate, urea, | | |
| | sodium chloride, lauryl | | |
| | diethylenediaminoglycerol, lauryl | | |
| | aminopropylglycerol, allantoin, SD alcohol | | |
| | 39-c (alcohol denat.) | | |
| | Uvinul P 25 | | 3.00% |
| | PEG-25 PABA Nipaguard ® CMB | (Clariant) | 0.15% |
| | Triethylene glycol, benzyl alcohol, propylene | | |
| | glycol, chloromethylisothiazolinone, | | |
| | methylisothiazolinone | | |
| E | Aminomethylpropanol | | 0.30% |

Preparation:

I Dissolve components C in B

II Stir components D successively into I

III Adjust the pH with E

IV Add A to III with stirring

### Examples 5A, 5B and 5C

### Skincare Compositions Without Oil

| | | | |
|---|---|---|---|
| A | Water | | ad 100% |
| B | Glycerol | | 3.00% |
| | Extrapon ® camomile special | | 1.00% |
| | Bisabolol extract | | |
| | Polyglycol 400 | (Clariant) | 4.50% |
| | PEG-8 | | |
| | Polyglycol 1500 S | (Clariant) | 1.50% |
| | PEG-32 | | |
| | Urea; chem. pure | | 2.00% |
| | Copolymer from example A, B or C | | 1.00% |
| | Fragrance | | 0.15% |
| | Nipaguard ® CMB | (Clariant) | 0.10% |
| | Triethylene glycol and benzyl alcohol and | | |
| | propylene glycol and | | |
| | chloromethylisothiazolinone and | | |
| | methylisothiazolinone | | |
| C | NaOH (20%) | | q.s. |

Preparation:

I Stir components B successively into A

II Adjust the pH with C

### Examples 6A, 6B and 6C

### O/W Skin Milk

| | | | |
|---|---|---|---|
| A | Hostacerin ® DGL | (Clariant) | 2.00% |
| | Polyglyceryl-2 PEG-10 laurate | | |
| | Isopropyl palmitate | | 4.00% |
| | Almond oil | | 5.00% |
| | Wheatgerm oil | | 1.00% |
| | Cetearyl isononanoate | | 8.00% |
| B | Copolymer from example A, B or C | | 0.80% |
| C | Water | | ad 100% |
| | Preservative | | q.s. |
| D | Fragrance | | 0.30% |

Preparation:

I Mix A and B, then stir into C

II Add D to I with stirring

III Homogenize the emulsion

### Examples 7A, 7B and 7C

### O/N Cream

| | | | |
|---|---|---|---|
| A | Hostacerin ® DGI | (Clariant) | 2.00% |
| | Polyglyceryl-2 sesquiisostearate | | |
| | Isopropyl palmitate | | 4.00% |
| | Octyldodecanol | | 4.00% |
| | Nipaguard ® PDU | (Clariant) | q.s. |
| | Propylene glycol (and) diazolidinylurea (and) | | |
| | methylparaben (and) propylparaben | | |
| B | Copolymer from Example A, B or C | | 1.20% |
| C | Hostapon ® CCG | (Clariant) | 0.80% |
| | Sodium cocoylglutamate | | |
| | Water | | ad 100% |
| D | Fragrance | | 0.40% |

Preparation:

I Stir B into A, then add C and stir

II Stir D into I

III Homogenize the emulsion

### Examples 8A, 8B and 8C

### O/W Day Cream with UVA and UVB Protection

| | | | |
|---|---|---|---|
| A | Hostaphat ® KW 340 D | (Clariant) | 1.0% |
| | Triceteareth-4 phosphate | | |
| | Glyceryl stearate | | 0.5% |
| | Cetearyl alcohol | | 0.5% |
| | Mineral oil, low-viscosity | | 7.0% |
| | Isopropyl palmitate | | 6.0% |
| | SilCare ® Silicone 41M15 | (Clariant) | 1.0% |
| | Caprylyl methicone | | |
| | Caprylic/capric triglycerides | | 2.0% |
| | Benzophenone-3 | | 1.0% |
| B | Copolymer from example A, B or C | | 1.0% |
| C | Water | | ad 100% |
| | Glycerol | | 5.0% |
| | Tinosorb ® M | | 3.0% |
| | Methylene bis-benzotriazolyl | | |
| | tetramethylbutylphenol | | |
| | Allantoin | (Clariant) | 0.3% |
| | Allantoin | | |
| D | Tocopheryl acetate | | 1.0% |
| | Fragrance | | 0.3% |
| | Nipaguard ® PDU | (Clariant) | q.s. |
| | Propylene glycol (and) diazolidinylurea (and) | | |
| | methylparaben (and) propylparaben | | |

Preparation:

I Melt A at about 70° C.

II Heat C to about 70° C.

III Stir B into A, then immediately add C and stir to cooling to about 30° C.

IV Stir D into III at about 30° C.

### Examples 9A, 9B and 9C

### Aftersun Cream Gel

| | | | |
|---|---|---|---|
| A | Mineral oil, low-viscosity | | 3.00% |
| | Isopropyl palmitate | | 3.00% |
| | Cetiol ® SN | | 3.00% |
| | Cetearyl isononanoate | | |
| | Jojoba oil | | 3.00% |
| | Walnut oil | | 3.00% |
| | Panthenol | | 1.00% |
| | Tocopheryl acetate | | 1.00% |
| B | Copolymer from example A, B or C | | 1.00% |
| C | Water | | ad 100% |
| | Glycerol | | 3.00% |
| | Allantoin | (Clariant) | 0.20% |
| | Allantoin | | |
| | Preservative | | q.s. |
| D | Collagen natural 1% | | 3.00% |
| | Alcohol | | 1.50% |
| | Fragrance | | 0.30% |

Preparation:

I Melt A at about 70° C., then add B

II Heat C to about 70° C.

III Stir II into I and cool to about 35° C. with stirring

IV Add D to III at about 35° C.

V Homogenize the emulsion

### Examples 10A, 10B and 10C: O/W Sun Protection Milk

| | | | |
|---|---|---|---|
| A | Aristoflex ® PEA | (Clariant) | 2.00% |
| | Polypropylene terephthalate | | |
| | Isopropyl palmitate | | 5.00% |
| | Mineral oil, perliquidium | | 10.00% |
| | Neo Heliopan ® E 1000 | | 8.50% |
| | Isoamyl p-methoxycinnamate | | |
| | Neo Heliopan ® BB | | 1.50% |
| | Benzophenone-3 | | |
| B | Copolymer from example A, B or C | | 0.60% |
| C | Water | | ad 100% |
| | Glycerol | | 3.00% |
| | Preservative | | q.s. |
| D | Fragrance | | 0.30% |

Preparation:

I Melt A at about 60° C., add B

II Add C, vigorous stirring

III Add D to II at about 35° C.

IV Homogenize the emulsion

### Examples 11A, 11B and 11C

### O/W Sun Protection Lotion, Sprayable

| | | | |
|---|---|---|---|
| A | Hostacerin ® DGI | (Clariant) | 4.00% |
| | Polyglyceryl-2 sesquiisostearate | | |
| | Eutanol ® G | | 4.50% |
| | Octyldodecanol | | |
| | Eusolex ® 2292 | | 10.00% |
| | Ethylhexyl methoxycinnamate | | |
| | Eusolex ® 9020 | | 5.00% |
| | Butyl methoxydibenzoylmethane | | |
| | Eusolex ® 6300 | | 4.00% |
| | 4-Methylbenzylidenecamphor | | |
| B | Copolymer from example A, B or C | | 0.50% |
| C | Water | | 40.00% |
| D | Water | | ad 100% |
| | Hostapon ® KCG | (Clariant) | 1.50% |
| | Sodium cocoylglutamate | | |
| | Glycerol | | 3.00% |
| | Panthenol | | 0.50% |
| E | Alcohol | | 5.00% |
| | Tocopheryl acetate | | 0.50% |
| | Fragrance | | q.s. |
| | Nipaguard ® PDU | (Clariant) | q.s. |
| | Propylene glycol (and) diazolidinylurea (and) | | |
| | methylparaben (and) propylparaben | | |

Preparation:

I Melt A at about 80° C.

II Add B to I

III Heat C to about 80° C.

IV Stir III into I with vigorous stirring (Ultraturrax/staro), about 2 minutes

V Very slowly stir a cold solution of D into IV

VI Stir E into V and stir for 1 hour

VII Homogenize the emulsion

### Examples 12A, 12B and 12C

### Sun Protection Cream

| | | | |
|---|---|---|---|
| A | Hostaphat ® KL 340 D | (Clariant) | 1.00% |
| | Trilaureth-4 phosphate | | |
| | Mineral oil, low-viscosity | | 8.00% |
| | Isopropyl palmitate | | 3.00% |
| | Myritol 318 | | 2.00% |
| | Caprylic/capric triglyceride | | |
| | Glyceryl stearate | | 0.50% |
| | Cetearyl alcohol | | 0.50% |
| B | Copolymer from Example A, B or C | | 0.80% |
| C | Glycerol | | 5.00% |
| | Alcohol | | 1.00% |
| | Water | | ad 100% |
| D | Tocopheryl acetate | | 1.00% |
| | Z-Cote HP1 | | 10.00% |
| | Zinc oxide and dimethicone | | |
| | Preservative | | q.s. |
| E | Fragrance | | 0.30% |

Preparation:

I Melt A at about 70° C., then add B

II Heat C to about 40° C.

III Stir II into I

IV Add D to III at about 35° C.

V Add C to IV

### Examples 13A, 13B and 13C

### Moisturizing Cream Gel

| | | | |
|---|---|---|---|
| A | Mineral oil, low-viscosity | | 6.00% |
| | Isopropyl palmitate | | 3.60% |
| | Soybean oil | | 2.40% |
| B | Copolymer from example A, B or C | | 1.00% |
| C | Water | | ad 100% |
| | Glycerol | | 8.00% |
| | Nipaguard ® PDU | (Clariant) | q.s. |
| | Propylene glycol (and) diazolidinylurea (and) | | |
| | methylparaben (and) propylparaben | | |
| D | Fragrance | | 0.30% |

Preparation:

I Mix A and B

II Stir C into I, then add D

II Homogenize the emulsion

### Examples 14A, 14B and 14C

### O/W Antiacne Skin Milk

| | | | |
|---|---|---|---|
| A | Hostacerin ® DGL | (Clariant) | 1.00% |
| | PEG-10 polyglyceryl-2 laurate | | |
| | Hostacerin ® DGSB | (Clariant) | 4.00% |
| | PEG-4 polyglyceryl-2 stearate | | |
| | Mineral oil, low-viscosity | | 5.00% |
| | Eutanol ® G | (Henkel) | 8.00% |
| | Octyldodecanol | | |
| | Isopropyl palmitate | (Uniqema) | 5.00% |
| | Jojoba oil | | 2.00% |
| B | Copolymer from example A, B or C | | 0.90% |
| C | Octopirox ® | (Clariant) | 0.20% |
| | Piroctone olamine | | |
| D | 1,2-Propylene glycol | | 10.00% |
| E | Water | | ad 100% |
| | Allantoin | (Clariant) | 0.20% |
| F | Preservative | | q.s. |
| | Perfume | | 0.30% |

Preparation:

I Melt A at about 60° C., then add B

II Dissolve C in heated D

II Stir II into I

IV Heat E to 60° C.

V Stir IV into III

VI Stir and cool to 35° C.

VII Add F to VI at about 35° C.

VIII Homogenize

### Examples 15A, 15B and 15C

### O/W Body Lotion

| | | | |
|---|---|---|---|
| A | Mineral oil, low-viscosity | | 4.00% |
| | Isopropyl palmitate | | 4.00% |
| | Eutanol ® G | | 4.00% |
| | Ethylhexyldodecanol | | |
| | Cetiol ® HE | | 0.50% |
| | PEG-7 glycerylcocoate | | |
| | Cetiol ® LC | | 0.50% |
| | Coco-caprylate/caprate | | |
| | SilCare Silicone ® 41M15 | (Clariant) | 0.50% |
| | Caprylyl methicone | | |
| B | Water | | ad 100% |
| | Glycerol | | 3.00% |
| | Panthenol | | 0.50% |
| | Polyglycol 35000 S | (Clariant) | 1.50% |
| | PEG-800 | | |
| | Allantoin | (Clariant) | 0.20% |
| | Allantoin | | |
| | Copolymer from example A, B or C | | 0.30% |
| C | Hostapon ® KCG | (Clariant) | 6.00% |
| | Sodium cocoylglutamate | | |
| | Fragrance | | 0.30% |
| D | Nipaguard ® PDU | (Clariant) | q.s. |
| | Propylene glycol (and) diazolidinylurea (and) | | |
| | methylparaben (and) propylparaben | | |

Preparation:

I Mix components A

II Dissolve B with stirring

III Add II to I with vigorous stirring, after about 5 minutes add C

IV After 5 minutes add D

### Examples 16A, 16B and 16C

### Mascara

| | | | |
|---|---|---|---|
| A | Tylose ® H 4000 G4 | (Clariant) | 0.70% |
| | Hydroxyethylcellulose | | |
| | 1,2-Propylene glycol | | 1.00% |
| | Water | | ad 100% |
| B | Triethanolamine 99% | | 1.20% |
| C | Stearic acid | | 3.00% |
| | SilCare ® Silicone 41M15 | (Clariant) | 1.00% |
| | Caprylyl methicone | | |
| | SilCare ® Silicone 31M40 | (Clariant) | 2.00% |
| | Caprylyl trimethicone | | |
| | Tegocare ® 450 | | 4.00% |
| | Polyglyceryl-3 methylglucose distearate | | |
| | Nexbase ® 2006 | | 2.00% |
| | Poly-1-decene | | |
| | Beeswax | | 2.50% |
| | Candelilla wax | | 2.50% |
| | Lunacera M | | 3.50% |
| | Microwax | | |
| D | Pigments | | 10.00% |
| E | Copolymer from example A, B or C | | 0.40% |
| F | Nipagin ® M | (Clariant) | 0.20% |
| | Methylparaben | | |
| | Nipasol ® M | (Clariant) | 0.10% |
| | Propylparaben | | |
| G | Fragrance | | q.s./optional |

Preparation:

I Heat a solution of Tylose H 4000 G4 and water to 85° C.

II Add B to I with vigorous stirring

III Mix components D and melt at about 85° C.

IV Add D and E to III, homogenize at about 85° C.

V Add II to IV with vigorous stirring, about 30 minutes

VI Add F to IV at about 35°-40° C.

### Examples 17A, 17B und 17C

### Hair Gel

| | | | |
|---|---|---|---|
| A | Genapol ® HS 200 | (Clariant) | 0.20% |
| | Steareth-20 | | |
| | Fragrance | | 0.20% |
| B | Water | | ad 100% |
| | Genapol ® HS 200 | (Clariant) | 1.80% |
| | Steareth 20 | | |
| C | Propylene glycol | | 2.00% |
| | Diaformer ® Z-651 | (Clariant) | 4.50% |
| | Acrylate/lauryl acrylate/stearyl acrylate/ | | |
| | ethylamine oxide methacrylate copolymer | | |
| | Alcohol denat. | | 20.00% |
| D | Dye | | q.s. |
| | Nipaguard ® MPA | (Clariant) | 0.50% |
| | Benzyl alcohol (and) methylparaben | | |
| | (and) propylparaben | | |
| E | Copolymer from example A, B or C | | 0.80% |

Preparation:

I Mix components A

II Dissolve components B with mild heating, cool and add to I

III Add components C successively to II

IV Add components D to III

V Add E to IV and homogenize

### Examples 18A, 18B and 18C

### Styling Fluid

| | | | |
|---|---|---|---|
| A | Sorbitol | | 5.00% |
| | Bozequat ® 4000 | (Clariant) | 0.30% |
| | Polyquaternium-43 | | |
| B | Water | | ad 100% |
| C | Copolymer from example A, B or C | | 2.00% |
| D | Aristoflex ® A 60 | (Clariant) | 5.00% |
| | VA/Crotonates copolymer | | |
| | Emulsogen ® HCO 040 | (Clariant) | 4.00% |
| | PEG-40 hydrogenated castor oil | | |
| E | Fragrance | | 0.20% |
| | Nipaguard ® DMDMH | (Clariant) | 0.30% |
| | DMDM hydantoin | | |
| | Dye | | q.s. |
| | Timiron Diamond Cluster MP-149 | | q.s. |
| | Mica and titanium dioxide EU: CI 77891) | | |
| F | Aminomethyl propanol | | 0.30% |

Preparation:

I Mix components A

II Add B to I

III Swell C in II with stirring

IV Mix components D and add IV to III

V Add components E successively to IV

VI Adjust to pH 7.0 with F

### Examples 19A, 19B und 19C

### Nail Varnish Remover, Gel

| | | | |
|---|---|---|---|
| A | Copolymer from example A, B or C | | 1.50% |
| B | Water | | ad 100% |
| C | Acetone | | 50.00% |
| | Glycerol | | 5.00% |
| | Polyglycol 400 | (Clariant) | 3.00% |
| | PEG-8 | | |

Preparation:

I Dissolve components C successively in B

II Stir A into I

### Examples 20A, 20B and 20C

### Glitter-Effect Shower Product

| | | | |
|---|---|---|---|
| A | Water | | ad 100% |
| B | Copolymer from example A, B or C | | 1.50% |
| C | Genapol ® LRO liquid | (Clariant) | 30.00% |
| | Sodium laureth sulfate | | |
| | Fragrance | | 0.50% |
| | Nipaguard ® DCB | (Clariant) | 0.10% |
| | Phenoxyethanol (and) | | |
| | methyldibromoglutaronitrile | | |
| D | Hostapon ® KCG | (Clariant) | 5.00% |
| | Sodium cocoylglutamate | | |
| E | Cirebelle 104 | | 0.20% |
| | Synthetic wax | | |

Preparation:

I Add A to B and stir to a homogeneous gel

II Mix components C and add to I with stirring, homogenize

III Add D to II

IV Add E to III

V Adjust pH

### Examples 21A, 21B and 21C

### Face Cleansing Lotion with Care Action

| | | | |
|---|---|---|---|
| A | Glycerol | | 8.00% |
| | Polyglycol 400 | (Clariant) | 5.00% |
| | PEG-8 | | |
| | Panthenol | | 0.50% |
| | Fragrance | | 0.20% |
| | Alcohol | | 8.00% |
| | Preservative | | q.s. |
| | Allantoin | (Clariant) | 0.10% |
| | Allantoin | | |
| | Niacinamide | | 0.10% |
| | Extrapon Hamamelis | | 1.00% |
| | Water, couch grass nut distillate, SD alcohol | | |
| | 39-C, butylene glycol | | |
| B | Water | | ad 100% |
| C | Copolymer from example A, B or C | | 0.30% |

Preparation:

I Dissolve A in B with stirring

II Add C to I with stirring and homogenize

### Examples 22A, 22B and 22C

### Refreshing Body Gel with Conditioning Action

| | | | |
|---|---|---|---|
| A | Water | | ad 100% |
| B | Alcohol denat. | | 20.00% |
| | Glycerol | | 5.00% |
| | Polyglycol 35000 S | (Clariant) | 0.50% |
| | PEG-800 | | |
| | Allantoin | (Clariant) | 0.20% |
| | Allantoin | | |
| | Copolymer from example A, B or C | | 1.00% |
| | Fragrance | | 0.15% |
| | Nipaguard ® MPA | (Clariant) | q.s. |
| | Benzyl alcohol (and) methylparaben (and) | | |
| | propylparaben | | |

Preparation:

Stir components B successively into A

### Examples 23A, 23B and 23C

### Sunscreen Gel

| | | |
|---|---|---|
| A | Ethanol | 50.00% |
| | Water | ad 100% |
| | Uvinul P25 | 6.00% |
| | PEG-25 PABA | |
| | Glycerol | 4.00% |
| | D-Panthenol | 1.00% |
| | Panthenol | |
| | Dow Corning 193 surfactant | 0.50% |
| | PEG-12 dimethicone | |
| B | Copolymer from example A, B or C | 1.00% |
| C | Fragrance | 0.30% |

Preparation:

I Stir B into A

II Add C

### Examples 24A, 24B and 24C

### O/V Sunscreen, Sprayable Foam

| | | | |
|---|---|---|---|
| A | Eusolex ® 2292 | | 8.00% |
| | Ethylhexyl methoxycinnamate | | |
| | Eusolex ® HMS | | 8.00% |
| | Homosalate | | |
| | Eusolex ® 9020 | | 4.00% |
| | Butylmethoxydibenzoylmethane | | |
| | Eusolex ® 6300 | | 4.00% |
| | 4-Methylbenzylidenecamphor | | |
| | Isopropyl palmitate | | 2.00% |
| | SilCare Silicone ® 41M15 | (Clariant) | 0.50% |
| | Caprylyl methicone | | |
| | Eutanol ® G | | 2.00% |
| | Octyldodecanol | | |
| B | Water | | ad 100% |
| | Glycerol | | 7.00% |
| | Panthenol | | 0.50% |
| | Polyglycol 35000 S | (Clariant) | 2.00% |
| | PEG-800 | | |
| | Copolymer from example A, B or C | | 0.40% |
| C | Hostapon ® KCG | (Clariant) | 12.00% |
| | Sodium cocoylglutamate | | |
| | Fragrance | | 0.30% |
| D | Nipa preservative | (Clariant) | q.s. |

Preparation:

I Mix components A, heat to 70° C.

II Dissolve B with stirring and heat to 70° C.

III Add II to I and stir vigorously

IV Add C at about 35° C.

V After 5 minutes, add D

### Examples 25A, 25B and 25C

### Handwash Gel

| | | |
|---|---|---|
| A | Copolymer from example A, B or C | 0.80% |
| | Isopropyl alcohol | 50.60% |
| B | Water | ad 100% |
| C | Imwitor ® 312 | 2.00% |
| | Glyceryl laurate | |
| | Propylene glycol | 2.00% |
| | Glycerol | 2.00% |
| D | Dye | q.s. |

Preparation:

I Dissolve components of A in B with stirring

II Add components C to I

III Add D to II

1. A cosmetic, dermatological and/or pharmaceutical composition comprising at least one copolymer A containing
a) 1% to 50% by weight of structural units originating from N-vinylcaprolactam,
b) 49.99% to 98.99% by weight of at least one repeating structural unit of the formula (1) 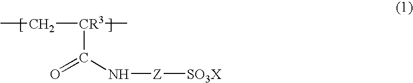
in which
R³ is hydrogen, methyl or ethyl,
Z is C₁-C₈ alkylene and
X⁺ is selected from the group consisting of Li⁺, Na⁺, K⁺, Mg²⁺/2, Ca²⁺/2, Al³⁺/3, NH₄⁺, monoalkylammonium, dialkylammonium, trialkylammonium or tetraalkylammonium, the alkyl substituents of the ammonium ions being independently of one another (C₁-C₂₂) alkyl radicals or (C₂-C₁₀) hydroxyalkyl radicals, singly to triply ethoxylated ammonium compounds having the same or different degree of ethoxylation, and mixtures thereof, and
c) 0.01% to 8% by weight of crosslinking structures originating from monomers having at least two olefinic double bonds.

2. The composition as claimed in claim 1, wherein the copolymers A contain 2% to 30% by weight of structural units originating from N-vinylcaprolactam, 69.5% to 97.5% by weight of structural units of the formula (1), derived in particular from the ammonium salt of 2-acrylamido-2-methylpropanesulfonic acid, and 0.2% to 4% by weight of crosslinking structures originating from monomers having at least two olefinic double bonds.

3. The composition as claimed in claim 1, wherein the crosslinking structures are structures originating from trimethylolpropane triacrylate.

4. The composition of claim 1, wherein the composition comprises 0.01% to 10% by weight of copolymers A.

5. The composition of claim 1, further comprising based on the total weight of the composition, 0.01% to 20% by weight of hydroxy acids or salts thereof.

6. The composition of claim 5, wherein the hydroxy acids are selected from the group consisting of glycolic acid, lactic acids, 2-hydroxyoctanoic acid, and mixtures thereof.

7. The composition of claim 1, which is in the form of an oil-in-water emulsion comprising based on the total weight of the composition,
a) up to 95% by weight of a water phase,
b) up to 40% by weight of an oil phase,
c) up to 15% by weight of one or more emulsifiers, and
d) up to 5% by weight of copolymer A.

8. The composition of claim 1, which is in the form of an oil-in-water gel cream comprising, based on the total weight of the composition,
a) up to 95% by weight of a water phase,
b) up to 30% by weight of an oil phase,
c) up to 5% by weight of one or more emulsifiers, and
d) up to 5% by weight of copolymer A.

9. The composition of claim 1, which is in the form of a water-in-oil emulsion comprising, based on the total weight of the composition,
a) up to 95% by weight of a water phase,
b) up to 60% by weight of an oil phase,
c) up to 20% by weight of one or more emulsifiers, and
d) up to 5% by weight of copolymer A.

10. The composition of claim 1, which is in the form of a water-in-silicone emulsion comprising, based on the total weight of the composition,
a) up to 90% by weight of a water phase,
b) up to 80% by weight of silicone oil,
c) 0.5% to 20% by weight of one or more emulsifiers, and
d) 0.01% to 5% by weight of copolymer A.

11. The composition of claim 1, which is in the form of a water-in-silicone emulsion comprising, based on the total weight of the composition,
a) up to 90% by weight of a water phase,
b) up to 80% by weight of silicone oil,
c) 0.5 to 20% by weight of one or more emulsifiers selected from the group consisting of cetyldimethicone copolyol, lauryldimethicone copolyol, PEG/PPG-18/18 dimethicone, trimethylsilylamodimethicone, and mixtures thereof, and
d) 0.01% to 5% by weight of copolymer A.

12. The composition of claim 1, further comprising one or more UV filter substances.

13. The composition of claim 1, further comprising one or more moisturizers or dyes or coloring pigments, or mixtures thereof.

14. The composition of claim 1, which is in the form of a suspension comprising, based on the total weight of the composition,
a) 0.1% to 10% by weight of copolymer A and
b) 0.1% to 30% by weight of solid particles, selected in from the group consisting of dyes, coloring pigments, effect pigments, light-protective pigments, adsorbents, abrasive components, and mixtures thereof.

15. The composition as claimed in claim 14, which is in the form of a gel-based eyeshadow comprising, based on the total weight of the composition,
a) 0.1% to 10% by weight of copolymer A and
b) 0.1% to 30% by weight of dyes or coloring pigments or mixtures thereof.

16. The composition of claim 1, which is in the form selected from the group consisting of a hair colorant, hair bleach, hair tinting composition, and mixtures thereof further comprising one or more substances selected from the group consisting of direct dyes, oxidation dye precursors, bleaching agents, and mixtures thereof.

17. The composition of claim 1 which is in the form of a hair bleach and further comprises one or more oxidizing agents.

18. The composition of claim 1, which is in the form of a deodorant or antiperspirant or a mixture thereof.

19. The composition of claim 1, which is in the form of a hair colorant, hair bleach, haircare or hair styling composition.

20. The composition of claim 1, which is in form selected from the group consisting of a pumpable form, a sprayable form, and a foamable form.

21. The composition as claimed in claim 20, containing no aerosol.

22. The composition of claim 1, wherein component c) is 0.01% to 5% by weight.

23. The composition as claimed in claim 1, wherein the copolymers A contain 3% to 15% by weight of structural units originating from N-vinylcaprolactam, 84.5% to 96.5% by weight of structural units of the formula (1), derived in particular from the ammonium salt of 2-acrylamido-2-methylpropanesulfonic acid, and 0.5% to 3% by weight of crosslinking structures originating from monomers having at least two olefinic double bonds.

24. The composition of claim 1, wherein the composition comprises 0.1% to 5% by weight of copolymers A.

25. The composition of claim 1, wherein the composition comprises 0.5% to 3% by weight of copolymers A.

26. The composition of claim 1, wherein the composition further comprises from 0.5% to 10% by weight of hydroxyl acids or a salt thereof.

27. The composition of claim 1, wherein the composition further comprises from 1% to 5% by weight of hydroxyl acids or salts thereof.

28. The composition of claim 5, wherein the hydroxyl acids or salts thereof are a-hydroxy acid or a salt thereof.

29. The composition of claim 1, which is in the form of an oil-in-water emulsion comprising, based on the total weight of the composition,
a) 60% to 92% by weight of a water phase,
b) 1% to 40% by weight of an oil phase,
c) 0.5% to 12% by weight of one or more emulsifiers, and
d) 0.01% to 5% by weight of copolymer A.

30. The composition of claim 1, which is in the form of an oil-in-water emulsion comprising, based on the total weight of the composition,
a) 70% to 90% by weight of a water phase,
b) 2% to 25% by weight of an oil phase,
c) 1% to 8% by weight of one or more emulsifiers, and
d) 0.05% to 3% by weight of copolymer A.

31. The composition of claim 1, which is in the form of an oil-in-water emulsion comprising, based on the total weight of the composition,
a) 75% to 85% by weight of a water phase,
b) 2% to 20% by weight of an oil phase,
c) 1% to 5% by weight of one or more emulsifiers, and
d) 0.1% to 2% by weight of copolymer A.

32. The composition of claim 1, which is in the form of an oil-in-water gel cream comprising, based on the total weight of the composition,
a) 50% to 95% by weight of a water phase,
b) 1% to 30% by weight of an oil phase,
c) 0.5% to 5% by weight of one or more emulsifiers, and
d) 0.01% to 5% by weight of copolymer A.

33. The composition of claim 1, which is in the form of an oil-in-water gel cream comprising, based on the total weight of the composition,
a) 70% to 90% by weight of a water phase,
b) 3% to 25% by weight of an oil phase,
c) 0.2% to 4% by weight of one or more emulsifiers, and
d) 0.05% to 3% by weight of copolymer A.

34. The composition of claim 1, which is in the form of an oil-in-water gel cream comprising, based on the total weight of the composition,
a) 75% to 85% by weight of a water phase,
b) 5% to 15% by weight of an oil phase,
c) 0.5% to 3% by weight of one or more emulsifiers, and
d) 0.1% to 2% by weight of copolymer A.

35. The composition of claim 1, which is in the form of an water-in-oil emulsion comprising, based on the total weight of the composition,
a) 40% to 95% by weight of a water phase,
b) 2% to 60% by weight of an oil phase,
c) 0.5% to 20% by weight of one or more emulsifiers, and
d) 0.01% to 5% by weight of copolymer A.

36. The composition of claim 1, which is in the form of an water-in-oil emulsion comprising, based on the total weight of the composition,
a) 50% to 95% by weight of a water phase,
b) 5% to 40% by weight of an oil phase,
c) 1% to 15% by weight of one or more emulsifiers, and
d) 0.05% to 3% by weight of copolymer A.

37. The composition of claim 1, which is in the form of an water-in-oil emulsion comprising, based on the total weight of the composition,
a) 60% to 85% by weight of a water phase,
b) 10% to 30% by weight of an oil phase,
c) 4% to 12% by weight of one or more emulsifiers, and
d) 0.1% to 2% by weight of copolymer A.

38. The composition of claim 1, which is in the form of a water-in-silicone emulsion comprising, based on the total weight of the composition,
a) 20% to 90% by weight of a water phase,
b) 10% to 70% by weight of silicone oil,
c) 1% to 15% by weight of one or more emulsifiers, and
d) 0.05% to 3% by weight of copolymer A.

39. The composition of claim 1, which is in the form of a water-in-silicone emulsion comprising, based on the total weight of the composition,
a) 40% to 85% by weight of a water phase,
b) 20% to 60% by weight of silicone oil,
c) 3% to 10% by weight of one or more emulsifiers, and
d) 0.1% to 2% by weight of copolymer A.

40. The composition of claim 1, which is in the form of a water-in-silicone emulsion comprising, based on the total weight of the composition,
a) 60% to 80% by weight of a water phase,
b) 30% to 50% by weight of silicone oil,
c) 1% to 15% by weight of one or more emulsifiers, and
d) 0.05% to 3% by weight of copolymer A.

41. The composition of claim 11, wherein the water-in-silicone comprises, based on the total weight of the composition, 20% to 90% by weight of component a), 10% to 70% by weight of component b), 1% to 15% by weight of component c), and 0.05% to 3% by weight of component d).

42. The composition of claim 11, wherein the water-in-silicone comprises, based on the total weight of the composition, 40% to 85% by weight of component a), 20% to 60% by weight of component b), 3% to 10% by weight of component c), and 0.1% to 2% by weight of component d).

43. The composition of claim 14, wherein the suspension comprises, based on the total weight of the composition, 0.2% to 6% by weight of component a), and 0.5% to 15% by weight of component b).

44. The composition of claim 14, wherein the suspension comprises, based on the total weight of the composition, 0.3% to 5% by weight of component a), and 1.0% to 10% by weight of component b).

45. The composition of claim 15, wherein the eyeshadow comprises 0.2% to 6% by weight of component a) and 0.5% to 15% by weight of component b).

46. The composition of claim 15, wherein the eyeshadow comprises 0.3% to 5% by weight of component a) and 1.0% to 10% by weight of component b).

47. The composition of claim 17, wherein the one or more oxidizing agents comprises hydrogen peroxide.
